(19) United States
(12) Patent Application Publication
(10) Pub. No.: US 2023/0402127 A1
(43) Pub. Date: Dec. 14, 2023

(54) MACHINE LEARNING-BASED VARIANT EFFECT ASSESSMENT AND USES THEREOF

(71) Applicant: Inari Agriculture Technology, Inc., Cambridge, MA (US)

(72) Inventors: Ross Everett ALTMAN, Boston, MA (US); Giles HALL, Medford, MA (US); Adam Patrick JOYCE, Stow, MA (US); Karl Anton Grothe KREMLING, Somerville, MA (US); Michael Andreas KOCK, Rheinfelden (DE)

(73) Assignee: Inari Agriculture Technology, Inc., Cambridge, MA (US)

(21) Appl. No.: 18/021,377

(22) PCT Filed: Jul. 6, 2021
(86) PCT No.: PCT/US21/40497

§ 371 Date: Feb. 14, 2023

Related U.S. Application Data
(60) Provisional application No. 63/068,687, filed on Aug. 21, 2020.

(51) Int. Cl. G16B 20/20; G16B 20/50; G16B 40/20; G06N 3/084

(52) U.S. Cl. CPC G16B 20/20; G16B 20/50; G16B 40/20; G06N 3/084

(57) ABSTRACT

Provided herein are machine learning-based methods for assessing the combined impact of multiple genetic variants, as well as the uses of such methods for various applications, such as in synthetic biology, personalized medicine, agricultural breeding, and genetic engineering. Also provided herein are exemplar computer-readable storage media and electronic devices for performing such methods.

### CROSS-REFERENCE TO RELATED APPLICATIONS

This application claims priority to the U.S. Provisional Patent Application No. 63/068,687 filed on Aug. 21, 2020, the entirety of which is incorporated herein by reference.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 165362000540SEQLIST.TXT, date recorded: Jul. 1, 2021, size: 16 KB).

### FIELD

The present disclosure relates generally to the field of genetics, and more specifically to the methods for using machine learning to assess the effects of genetic variants and uses thereof.

### BACKGROUND

A genetic variant refers to a nucleotide or polypeptide sequence that differs from a reference sequence for a given region. For example, a genetic variant may comprise a deletion, substitution, or insertion of one or more nucleotides or amino acids encoded thereof. Genetic variants are an important factor contributing to variation in a phenotype (e.g., a human disease or crop or livestock performance), and thus efficient and effective assessment of genetic variant effects are of significant importance to genetic and medical research. Recently, technological advances in high-throughput sequencing have greatly facilitated comprehensive investigations into the number and types of sequence variants possessed by individuals in different populations across phenotypes.

Prediction of the phenotypic consequences of genetic variants is the basis of various applications in a number of fields, including, for example, designing and synthesizing bio-molecules synthetic biology, prioritizing functional cancer-driving mutations to guide drug and chemotherapy choice in personalized genetic medicine, identifying antibiotic and antiviral susceptibilities of pathogens after repeated cycles of mutation and exposure to antimicrobials, and identifying transgenic or genome editing targets for plant and animal breeding. Lately, the use of computational algorithms and methods has aided in the prediction of the phenotypic effects of genetic variants. In the fields described above however, commonly used tools for variant effect prediction of genetic variants are inherently limited because they consider perturbation to only one site at a time relative to a reference sequence. Examples of these tools include PolyPhen & PolyPhen-2 (Adzhubei et al. 2010), SIFT (Ng et al, 2003), Provean (Choi et al. 2012), and GERP (Davydov et al. 2010). Because these tools focus first on conservation at the site level instead of predicting how a coding sequence variant might compromise a protein's biochemical function, they are inherently limited to only predicting the impact of one variant at a time.

Accordingly, there is a need for improved methods and computational tools for assessing genetic variants. Particularly, there is a need for using advanced computational methods, such as machine learning, to improve the accuracy and efficiency of predicting effects of pairwise and higher order interacting genetic variants.

### BRIEF SUMMARY

Provided herein are machine learning-based methods for assessing the combined impact of multiple genetic variants, as well as the uses of such methods for various applications, such as in synthetic biology, personalized medicine, agricultural breeding, and genetic engineering. Also provided herein are exemplar computer-readable storage media and electronic devices for performing such methods.

In one aspect, provided herein is a method for assessing effects of genetic variants, comprising: a) receiving a dataset of sequences from an input device, wherein the dataset of sequences comprises a reference sequence, a primary genetic variant of the reference sequence, and one or more secondary genetic variants in the reference sequence; b) automatically inputting the dataset of sequences to a trained machine-learning model to obtain one or more effect scores, wherein the model is configured to output one or more effect scores corresponding to the probabilities of one or more secondary genetic variants having a compensatory effect to the primary genetic variant, or the magnitudes of said effects; and c) displaying the predicted effect scores on a display device.

In some embodiments, the model is trained by: a) a pre-training task, comprising: 1) receiving a pre-training dataset comprising a plurality of batches of naturally occurring sequences; 2) inputting each batch of sequences into a language model, wherein the model is configured to output a pre-training set of semantic features; 3) automatically updating the language model after each batch; b) optionally, a fine-tuning task, comprising: 1) receiving a fine-tuning dataset comprising a plurality of batches of naturally occurring sequences, wherein the fine-tuning dataset is a subset of the pre-training dataset, or a set of sequences that are related to the pre-training dataset by common ancestry, homology, or multiple sequence alignment; 2) inputting each batch of sequences into the language model, wherein the model is configured to output a fine-tuning set of semantic features; and 3) automatically updating the language model after each batch; and c) a transfer learning task, comprising: 1) receiving a final training dataset comprising labeled sequences mapped to effects; and 2) training a neural network model based on the final training dataset, wherein the neural network model is configured to receive data corresponding to the pre-training set of semantic features and/or the fine-tuning set of semantic features, and output one or more effect scores.

In some other embodiments, the model is trained by: a) receiving a training dataset of sequences, comprising a training reference sequence and a training primary genetic variant, wherein the training primary genetic variant has an effect on the reference sequence with respect to a metric of interest; b) inputting the training dataset into a generative procedure configured to generate one or more training secondary genetic variants according to a random seed; c) calculating a loss function, wherein the loss function maps the combined effect of the primary and secondary genetic variants and the effect of the reference sequence onto a quantitative error score; d) accepting or rejecting the one or more training secondary genetic variants according to one or more predetermined acceptance criteria on the loss function; e) updating the generative procedure by incorporating the accepted one or more training secondary genetic variants in a new round of additional training secondary genetic variants; and f) repeating steps b) to e) until the loss converges to a minimum.

In some embodiments, the true compensatory effect is obtained from a saturation mutagenesis analysis. In some embodiments, the loss function is a binary loss function. In some embodiments, the loss function is based on a distance metric.

In some embodiments that may be combined with any of the preceding embodiments, the method further comprises selecting one or more secondary genetic variants based on the effect scores. In some embodiments, the method further comprises prioritizing one or more secondary genetic variants based on the effect scores. In some embodiments, the method further comprises evaluating epistasis of one or more secondary genetic variants based on the effect scores.

In some embodiments that may be combined with any of the preceding embodiments, the method further comprises: a) altering one or more of the secondary genetic variants in the genome of an organism; b) identifying an impact of the alteration on an endophenotype, wherein the endophenotype is a quantifiable phenotype at a sub-organismal level that can be measured by a biochemical, gene expression, or protein level assay; and c) updating the model using the identified endophenotypic impact.

In some embodiments that may be combined with any of the preceding embodiments, the genetic variant is an allele or a mutation as compared to the reference sequence. In some embodiments, the primary genetic variant is a deleterious genetic variant having a deleterious or disease-causing effect as compared to the reference sequence. In some embodiments, the primary genetic variant is a beneficial genetic variant having a beneficial or disease-preventing effect as compared to the reference sequence.

In some embodiments that may be combined with any of the preceding embodiments, the dataset of sequences are clustered by sequence similarity. In some embodiments, the dataset of sequences is obtained from a sequence database. In some embodiments, the sequence database is the UniRef database, the UniParc database, the UniProt database, the Pfam database, or the SwissProt database. In some embodiments, the dataset of sequences are DNA sequences, RNA sequences, or protein sequences. In some embodiments, the dataset of sequences are sequences from a single gene or a protein encoded thereby. In some embodiments, the dataset of sequences are sequences from a single gene family or a protein family encoded thereby. In some embodiments, the dataset of sequences are sequences from different genes or proteins encoded thereby, wherein the encoded proteins physically interact to form a complex. In some embodiments, the dataset of sequences are sequences from different components within a virus, an organelle, a cell, a tissue, an organ, or an organism. In some embodiments, the dataset of sequences are viral sequences, bacterial sequences, algal sequences, fungal sequences, plant sequences, animal sequences, or human sequences. In some embodiments, the dataset of sequences are from one or more coronaviruses. In some embodiments, the dataset of sequences are from one or more cancer cells.

In some embodiments that may be combined with any of the preceding embodiments, the effect is an effect at a molecular level, a cellular level, a sub-organismal level, or an organismal level. In some embodiments, the effect is an effect affecting an endophenotype selected from a group consisting of messenger RNA (mRNA) abundance, gene transcript splicing ratio, protein abundance, micro RNA (miRNA) or small RNA (siRNA) abundance, translational efficiency, ribosome occupancy, protein modification, metabolite abundance, and allele specific expression (ASE). In some embodiments, the effect is an effect affecting a protein property. In some embodiments, the effect is an effect affecting protein structure, protein conformation, protein molecular or cellular function, protein stability, protein solvent accessibility, enzymatic affinity, or enzymatic efficiency. In some embodiments, the effect is a collection of effects characterizing the state of a protein. In some embodiments, the effect is an effect affecting fitness of an organism with respect to either a specific environment or spanning a wide range of environments. In some embodiments, the effect is interpretable to humans and/or machines.

In another aspect, provided herein is a method for designing a molecule with a desired effect, comprising: a) receiving a dataset of sequences from an input device, wherein the dataset of sequences comprises a reference sequence, a primary genetic variant of the reference sequence, and one or more secondary genetic variants in the reference sequence; b) automatically inputting the dataset of sequences to a trained machine-learning model to obtain one or more effect scores, wherein the model is configured to output one or more effect scores corresponding to the probabilities of one or more secondary genetic variants having a compensatory effect to the primary genetic variant, or the magnitudes of said effects; c) displaying the predicted effect scores on a display device; and d) designing a molecule based on the effect scores. In some embodiments, the method further comprises synthesizing the designed molecule. In some embodiments, the effect of the designed molecule is stability, solubility, affinity, biological activity, bioavailability, binding specificity, subcellular localization, tissue-specific expression, a chemical property, a physical property, or a structural property. In some embodiments, the designed molecule is a DNA molecule, an RNA molecule, a protein molecule, or a complex of protein molecules. In some embodiments, the designed molecule is a single stranded DNA (ssDNA) or a double stranded DNA (dsDNA). In some embodiments, the designed molecule is a messenger RNA (mRNA), a transfer RNA (tRNA), a ribosomal RNA (rRNA), a small RNA (sRNA), or a guide RNA (gRNA). In some embodiments, the designed molecule is an antibody, a contractile protein, an enzyme, a hormonal protein, a structural protein, a storage protein, or a transport protein. In some embodiments, the designed molecule is a viral molecule, a bacterial molecule, an algal molecule, a fungal molecule, a plant molecule, an animal molecule, or a human molecule. In some embodiments, the designed molecule is a virus protein. In some embodiments, the virus protein is a protein from a coronavirus. In some embodiments, the coronavirus is a severe acute respiratory syndrome coronavirus 2 (SARS-CoV-2) that is the causal agent for the infectious disease coronavirus disease 2019 (COVID-19).

In yet another aspect, provided herein is a method for providing personalized and probabilistic information for a patient, comprising: a) receiving a dataset of sequences associated with a patient from an input device, wherein the dataset of sequences comprises a reference sequence, a primary genetic variant of the reference sequence, and one or more secondary genetic variants of the reference sequence; b) automatically inputting the dataset of sequences to a trained machine-learning model to obtain one or more effect scores, wherein the model is configured to output one or more effect scores corresponding to the probabilities of one or more secondary genetic variants having a compensatory effect to the primary genetic variant, or the magnitudes of said effects; c) displaying the predicted effect scores on a display device; and d) assisting in selection of one or more medical choices specific to the patient based on the effect scores. In some embodiments, the attribute associated with the patient is selected from the group consisting of genetic profile, predisposition or response to a disease, and response to a treatment. In some embodiments, the genetic profile is from one or more cancer tumors of the patient. In some embodiments, the disease is selected from the group consisting of cancer, obesity, hypertension, a cardiovascular disease, an infectious disease, an autoimmune disease, a genetic disease, a liver disease, insulin resistance, Crohn's disease, dementia, Alzheimer's disease, cerebral infarction, hemophilia, viral hepatitis, sickle cell disease, multiple sclerosis, and muscular dystrophy. In some embodiments, the treatment is selected from the group consisting of drug administration, chemotherapy, radiation therapy, immunotherapy, and gene therapy. In some embodiments, the one or more medical choices is selected from the group consisting of prognosis, diagnosis, treatment, intervention, and prevention.

In still another aspect, provided herein is a method for predicting resistance of a pathogen to an anti-pathogen treatment, comprising: a) receiving a dataset of sequences associated with a pathogen from an input device, wherein the dataset of sequences comprises a reference sequence, a primary genetic variant of the reference sequence, and one or more secondary genetic variants in the reference sequence; b) automatically inputting the dataset of sequences to a trained machine-learning model to obtain one or more effect scores, wherein the model is configured to output one or more effect scores corresponding to the probabilities of one or more secondary genetic variants having a compensatory effect to the primary genetic variant, or the magnitudes of said effects, wherein the effect affects an attribute associated with the pathogen having resistance to an anti-pathogen treatment; and c) displaying the predicted effect scores on a display device, corresponding to the predicted resistance of the pathogen to the anti-pathogen treatment. In some embodiments, the pathogen is a virus, a prion, a viroid, a bacterium, a fungus, a protozoan, or a parasite. In some embodiments, the attribute associated with the pathogen is selected from the group consisting of nucleic acid replication, DNA integration into a host genome, gene expression, protein synthesis, metabolism, cell membrane synthesis, cell wall synthesis, and peptidoglycan biosynthesis. In some embodiments, the anti-pathogen treatment is administering a drug selected from the group consisting of an antiviral, an antibacterial, an antibiotic, an antifungal, an antiparasitic, and a pesticide. In some embodiments, the pathogen is Neisseria gonorrhea, and the anti-pathogen treatment is administration of ciprofloxacin or ceftriaxone.

In still another aspect, provided herein is a method for identifying targets for genetically improving a trait in an organism, comprising: a) receiving a dataset of sequences associated with an organism from an input device, wherein the dataset of sequences comprises a reference sequence, a primary genetic variant of the reference sequence, and one or more secondary genetic variants in the reference sequence; b) automatically inputting the dataset of sequences to a trained machine-learning model to obtain one or more effect scores, wherein the model is configured to output one or more effect scores corresponding to the probabilities of one or more secondary genetic variants having a compensatory effect to the primary genetic variant, or the magnitudes of said effects, and wherein the effect affects an attribute associated with a trait of the organism; and c) displaying the predicted effect scores on a display device, corresponding to the targets for genetically improving the organism. In some embodiments, the method further comprises selecting one or more of the identified targets for genetic improvement of the organism. In some embodiments, the method further comprises selecting an organism with the improved trait. In some embodiments, the genetic improvement is achieved by conventional breeding. In some embodiments, the genetic improvement is achieved by a transgenic technology or a genome editing technology. In some embodiments, the genome editing technology is a base editing technology using a DNA base editor or an RNA base editor. In some embodiments, the genome editing is achieved by a clustered regularly interspersed short palindromic repeats (CRISPR) system, a transcription activator-like effector nuclease (TALEN) system, or a zinc finger nuclease (ZFN) system. In some embodiments, the genome editing is achieved by coupling with a recombination system. In some embodiments, the recombination system is a lambda phage derived recombination (lambda Red) system. In some embodiments, the organism is maize, wheat, barley, oat, rice, soybean, oil palm, safflower, sesame, tobacco, flax, cotton, sunflower, pearl millet, foxtail millet, sorghum, canola, cannabis, a vegetable crop, a forage crop, an industrial crop, a woody crop, or a biomass crop. In some embodiments, the trait is yield, overall fitness, biomass, photosynthetic efficiency, nutrient use efficiency, heat tolerance, drought tolerance, herbicide tolerance, or disease resistance. In some embodiments, the organism is cattle, sheep, goat, horse, pig, chicken, duck, goose, rabbit, or fish. In some embodiments, the trait of the organism is growth rate, feed use efficiency, meat yield, meat quality, milk yield, milk quality, egg yield, egg quality, wool yield, or wool quality. In some embodiments, provided herein is an organism genetically improved by the method of any of the preceding embodiments.

In still another aspect, provided herein is a method for identifying genetic variants as alternative candidates for use as targets in genome editing, comprising: a) receiving a dataset of sequences associated with an organism from an input device, wherein the dataset of sequences comprises a reference sequence, a primary genetic variant of the reference sequence, and two or more secondary genetic variants in the reference sequence; b) automatically inputting the dataset of sequences to a trained machine-learning model to obtain one or more effect scores, wherein the model is configured to output one or more effect scores corresponding to the probabilities of one or more secondary genetic variants having a compensatory effect to the primary genetic variant, or the magnitudes of said effects; and c) displaying the predicted effect scores on a display device, corresponding to the genetic variants as alternative candidates for use as targets in genome editing. In some embodiments, the method further comprises altering the identified genetic variants as alternative candidates targets that are more easily accessible by a transgenic technology or a genome editing technology. In some embodiments, the genome editing is achieved by a base editing technology using a DNA base editor or an RNA base editor. In some embodiments, provided herein is a base editing technology according to the method of any of the preceding embodiments.

In still another aspect, provided herein is a non-transitory computer-readable storage medium storing one or more programs, the one or more programs comprising instructions, which when executed by one or more processors of an electronic device having a display, cause the electronic device to: a) receive a dataset of sequences from an input device, wherein the dataset of sequences comprise a reference sequence, a primary genetic variant of the reference sequence, and one or more secondary genetic variants in the reference sequence; b) automatically input the dataset of sequences to a trained machine-learning model to obtain one or more effect scores, wherein the model is configured to output one or more effect scores corresponding to the probabilities of one or more secondary genetic variants having a compensatory effect to the primary genetic variant, or the magnitudes of said effects; and c) display the predicted effect scores on a display device. In some embodiments, the model is a discriminative model or a generative model.

In still another aspect, provided herein is an electronic device, comprising: a display; one or more processors; a memory; and one or more programs, wherein the one or more programs are stored in the memory and configured to be executed by the one or more processors, the one or more programs including instructions for: a) receiving a dataset of sequences from an input device, wherein the dataset of sequences comprise a reference sequence, a primary genetic variant of the reference sequence, and one or more secondary genetic variants in the reference sequence; b) automatically inputting the dataset of sequences to a trained machine-learning model to obtain one or more effect scores, wherein the model is configured to output one or more effect scores corresponding to the probabilities of one or more secondary genetic variants having a compensatory effect to the primary genetic variant, or the magnitudes of said effects; and c) displaying the predicted effect scores on a display device.

### DESCRIPTION OF THE FIGURES

FIG. 1A illustrates a diagram of an exemplary process for using machine learning to identify compensatory secondary genetic variants.

FIG. 1B illustrates the compensatory effect of a secondary genetic variant (e.g., a mutation) on maintaining proper and stable protein folding. The top row shows a wild-type (WT) gene model and the encoded properly folded protein, as well as the four potential mutation loci 1-4 on the WT gene model. The six gene models below the WT gene model show the various mutations (marked as “X”s) across mutation loci 1-4 on the WT. A triangle (Δ) at the site indicates the mutation, either alone or with other mutation(s) present in the same gene, does not affect proper and stable folding of the protein, i.e., having a non-pathogenic impact on the protein. A circle (O) at the site indicates the mutation, either alone or with other mutation(s) present in the same gene, prevents proper and stable folding of the protein, i.e., having pathogenic impact on the protein. The gene model on the bottom shows two mutations at locus 1 and locus 3 as a pair of compensatory mutations that lead to normal folding of the protein.

FIG. 2 illustrates a diagram of an exemplary training process with learning transfer for use with the methods of the present disclosure. Step (a) comprises a pre-training task using self-supervised next token prediction. Step (b) comprises a fine-tuning task using self-supervised next token prediction. Step (c) comprises a transfer learning task using supervised regression/classification.

FIG. 3 illustrates a diagram of an exemplary generative modeling-based training process for use with the methods of the present disclosure.

FIG. 4 illustrates a diagram of an exemplary method for designing a molecule with a desired effect.

FIG. 5 illustrates a diagram of an exemplary method for providing personalized and probabilistic information for a patient.

FIG. 6 illustrates a diagram of an exemplary method for predicting resistance of a pathogen to an anti-pathogen treatment.

FIG. 7 illustrates a diagram of an exemplary method for identifying targets for genetically improving a trait within an organism.

FIG. 8 illustrates a diagram of an exemplary method for identifying genetic variants as alternative candidates for use as more accessible targets in genome editing.

FIG. 9 illustrates an exemplary electronic device in accordance with some embodiments.

FIG. 10A and FIG. 10B show examples of identifying compensatory genetic variants using methods of the present disclosure, in the BBS4 protein (FIG. 10A) and RPGRIP1L protein (FIG. 10B). The upper panel of FIG. 10A shows the polypeptide sequence of BBS4 protein (SEQ ID NO: 1), with the primary genetic variant N/H variant in bold font at amino acid location 165, and the lower panel of FIG. 10A shows a series of compensatory variant pairs including the N165H/H366R pair that produces one of the least differences in protein stability compared to the wild-type protein (“Δ Protein Stability”). The upper panel of FIG. 10B shows the polypeptide sequence of the RPGRIP1L protein (SEQ ID NO: 2), with the primary genetic variant R/L, variant in bold font at amino acid location 937, and the lower panel of FIG. 10B shows a series of compensatory variant pairs including the R937L/R961 pair that produces one of the least differences in protein stability compared to the wild-type protein (“Δ Protein Stability”). The identified compensatory genetic variants are also compared with those identified by Jordan et al. (Nature 524, 225-229 (2015)) and functionally validated using the model organism zebrafish, where a set of potential compensating variants are investigated and the top-k candidates are identified (with k=2 for BBS4 and k=3 for RPGRIP1L). Results show that at least one of the top-k compensating variants identified by the methods of the present disclosure (shown in solid box) agrees with the findings from the zebrafish study.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown but are to be accorded the scope consistent with the claims.

Although the following description uses terms “first”, “second”, etc. to describe various elements, these elements should not be limited by the terms. These terms are only used to distinguish one element from another. For example, a first graphical representation could be termed a second graphical representation, and, similarly, a second graphical representation could be termed a first graphical representation, without departing from the scope of the various described embodiments. The first graphical representation and the second graphical representation are both graphical representations, but they are not the same graphical representation.

The terminology used in the description of the various described embodiments herein is for the purpose of describing particular embodiments only and is not intended to be limiting. As used in the description of the various described embodiments and the appended claims, the singular forms “a”, “an”, and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes”, “including”, “comprises”, and/or “comprising”, when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting”, depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]”, depending on the context.

The present invention is based, at least in part, on the surprising results that increased effectiveness and efficiency of predicting the effects of pair-wise and higher-order interacting genetic variants are achieved by using the machine learning-based methods disclosed herein. Accordingly, provided herein are machine learning-based methods for assessing the combined impact of multiple genetic variants, as well as the uses of such methods for various applications, such as in synthetic biology, personalized medicine, agricultural breeding, and genetic engineering. Also provided herein are exemplar computer-readable storage media and electronic devices for performing such methods.

In the fields of human genetics and medicine, commonly used tools for variant effect prediction of genic variants are inherently limited because they consider perturbation to only one site at a time relative to a reference genome. Examples of these tools include PolyPhen & PolyPhen-2 (Adzhubei et al. 2010), SIFT (Ng et al. 2003), Provean (Choi et al. 2012) and GERP (Davydov et al. 2010). Because these tools focus first on conservation at the site level instead of predicting how a coding sequence variant might compromise a protein's biochemical function, they are inherently limited to only predicting the impact of one site at a time. However, they do not account for multiple potentially compensatory or synergistically negative variants co-occurring in the same haplotype or within cells that, for example, comprise a tumor. This is particularly relevant in cancers for which the tumor comprises a population of genetically heterogeneous cells, which are themselves continuously mutating. In these tumors, the functional cancer driver mutation(s) may be changing because of mutation and selection after administration of a chemotherapeutic drug, and, thus, tools that can better predict the impact of multiple mutations in a complex tumor can better determine which mutations are truly the driver mutations. This lack of consideration of local epistasis (e.g., interplay among physically interacting mutations) leads to misclassification of functionally benign mutations as pathogenic and classification of pathogenic mutations as benign. The consequence of this misclassification of variants is greater false positive and false negative rates leading to misdiagnosis of disease or carrier status and potential incorrect prescription of treatment. In addition, in cases where individual mutations are being prioritized for editing in a plant or animal improvement program, these false positive targets whose deleterious impacts have already been complemented by another mutation or mutations at a nearby local site mean that an edit may in fact lead to a loss of fitness.

Compared to commonly used methods or tools for variant effect prediction, the methods described herein have improved accuracy and efficiency in predicting effects of pairwise and higher order interacting genetic variants, including for example, within a protein or known complex. The methods of the present disclosure allow for the prediction of protein function directly from nucleotide or amino acid sequence and enable assessment of higher order combinations of disrupting and compensating variants within proteins, resulting in more accurate assessment of which variants are functional conditioned on the presence of other variants.

Uses of the disclosed methods include not only local compensatory coding sequence variants in the same gene or in a complex, but also compensating regulatory variations. In other words, if a variant is predicted to reduce a protein's stability and it co-occurs with a cis variant that appears to increase expression to compensate, this could assist in determining which of the coding sequence variants is indeed functionally deleterious.

Additional applications of the disclosed methods include utility in single cell cancer genome profiling given that then one can tell from a heterogeneous sample if compensatory variants co-occur in the same source cell's genome or if the putative compensatory variants occur in separate genomes. The methods described herein can also be used for predicting effects of pairwise or higher order combinations of variants in different genes when there is a known physical interaction between the encoded proteins, such as those in KRAS and EGFR (Wilkins et al. 2018).

### Machine Learning-Based Variant Effect Assessment

Accordingly, in one aspect, provided herein is a method for assessing effects of genetic variants, comprising: a) receiving a dataset of sequences from an input device, wherein the dataset of sequences comprises a reference sequence, a primary genetic variant of the reference sequence, and one or more secondary genetic variants in the reference sequence; b) automatically inputting the dataset of sequences to a trained machine-learning model to obtain one or more effect scores, wherein the model is configured to output one or more effect scores corresponding to the probabilities of one or more secondary genetic variants having a compensatory effect to the primary genetic variant, or the magnitudes of said effects; and c) displaying the predicted effect scores on a display device.

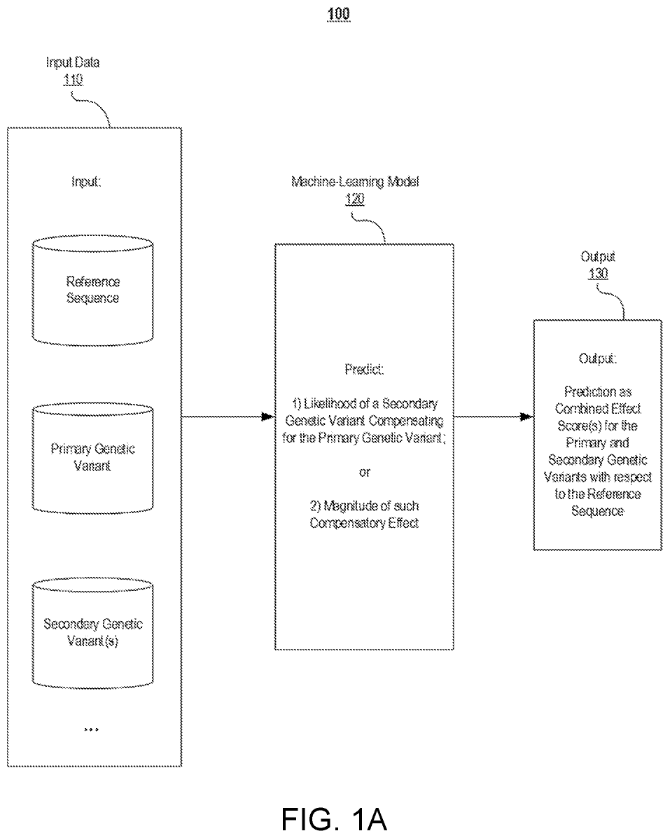

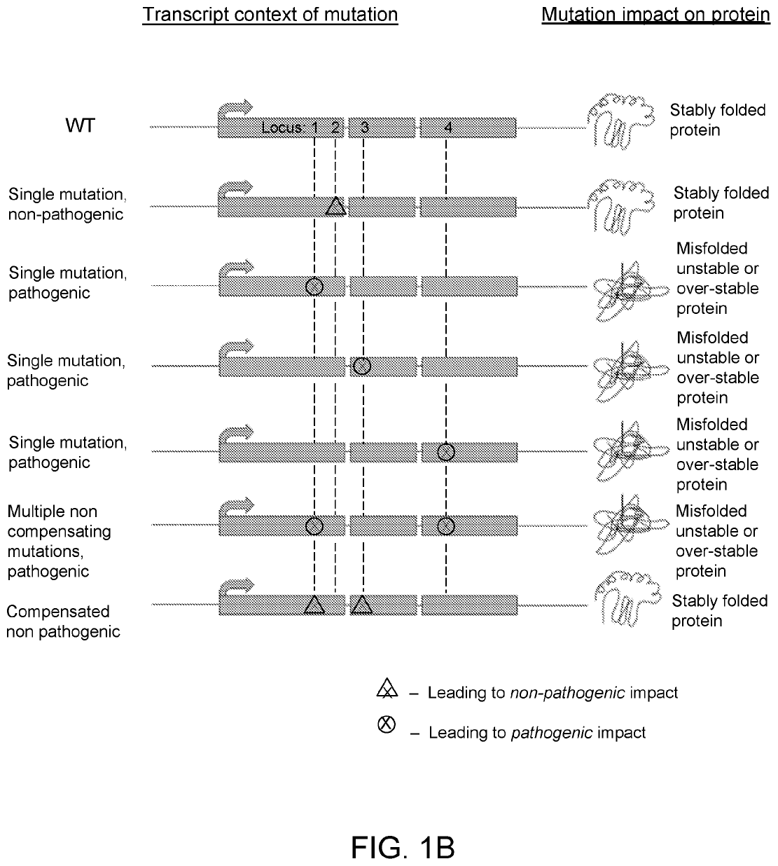

FIG. 1A illustrates a diagram of an exemplary process 100 for using machine learning to identify compensatory secondary genetic variants, in accordance with some embodiments of the present disclosure. Generally, the input data 110 is passed onto the machine learning model 120, which is configured to output one or more effect scores 130 corresponding to the probabilities of one or more secondary genetic variants having a compensatory effect to the primary genetic variant, or the magnitudes of said effects.

### Input Sequences

With reference to FIG. 1A, the inputting dataset of sequences 110 comprises a reference sequence, a primary genetic variant, and one or more secondary genetic variants.

In some embodiments, the sequences are obtained from a sequence database. Various suitable nucleotide or polypeptide sequence databases are known in the art and may be used with the methods described herein. Examples of publicly available sequence databases include, but are not limited to, GenBank, EMBL, DDBJ, RefSeq, PIR, PRF, TPA, PDB, Pfam, UnitProt (including, for example, UniRef, UniParc, UniProtKB/Swiss-Prot, and UniProtKB/TrEMBL). In some embodiments, the sequence database is the UniRef database, the UniParc database, the UniProt database, the Pfam database, or the SwissProt database. In some embodiments, the dataset of sequences are clustered by sequence similarity.

The terms “sequence similarity” and “sequence identity” with respect to a nucleic acid sequence are defined as the percentage of nucleotides in a candidate sequence that are identical with the nucleotides in the specific nucleic acid sequence, after aligning the sequences by allowing gaps, if necessary, to achieve the maximum percent sequence identity. The terms “sequence similarity” and “sequence identity” with respect to a peptide, polypeptide or protein sequence refer to the percentage of amino acid residues in a candidate sequence that are identical substitutions to amino acid residues in the specific peptide or amino acid sequence, after aligning the sequences by allowing gaps, if necessary, to achieve the maximum percent sequence homology. Alignment for purposes of determining percent sequence identity can be achieved in various ways that are within the skill in the art, for instance, using publicly available computer software such as BLAST, BLAST-2, ALIGN, or MEGALIGN™ (DNASTAR) software. Those skilled in the art can determine appropriate parameters for measuring alignment, including any algorithms needed to achieve maximal alignment over the full length of the sequences being compared.

The input sequences of the present disclosure may be of various types and/or from various origins. In some embodiments, the dataset of sequences are DNA sequences, RNA sequences, or protein sequences. In some embodiments, the dataset of sequences are sequences from a single gene or a protein encoded thereby. In some embodiments, the dataset of sequences are sequences from a single gene family or a protein family encoded thereby. In some embodiments, the dataset of sequences are sequences from different genes or proteins encoded thereby, wherein the encoded proteins physically interact to form a complex. In some embodiments, the dataset of sequences are sequences from different components within a virus, an organelle, a cell, a tissue, an organ, or an organism. In some embodiments, the dataset of sequences are viral sequences, bacterial sequences, algal sequences, fungal sequences, plant sequences, animal sequences, human sequences, or sequences from a particular phylogenetic lineage. In some embodiments, the dataset of sequences are from one or more coronaviruses. In some embodiments, the dataset of sequences are from one or more cancer cells.

Genetic Variants

As used herein, the terms “genetic variant” and “variant” refer to a nucleotide or polypeptide sequence that differ from a reference sequence for a given region. For example, a genetic variant may comprise a deletion, substitution, or insertion of one or more nucleotides or amino acids encoded thereof. When the reference sequence refers to a normal or wild-type sequence, a genetic variant may also be referred to as a “mutation” and an organism having such mutation as a “mutant.” When it is used in the context of an alternative form of a sequence, especially that of a gene in a population, a genetic variant may also be referred to as an “allele.” Accordingly, in some embodiments, the genetic variant of the present disclosure is allele. In some embodiments, the genetic variant is a mutation.

Various types of genetic variants may be used with the methods of the present disclosure, which include, for example, frameshift, stop gained, start lost, splice acceptor, splice donor, stop lost, inframe indel, missense, splice region, synonymous, and copy number variants. Non-limiting types of copy number variants include deletions and duplications. The genetic variants in the present disclosure may be provided by comparing different sequences at a given region. Methods and techniques of sequencing and sequence alignment are known in the art. See e.g., Adams et al., eds. Automated DNA sequencing and analysis. Elsevier, 2012, França et al., Quarterly reviews of biophysics, 35(2), 169-200, and Rosenberg, M. S. ed., 2009. Sequence alignment: methods, models, concepts, and strategies. Univ of California Press.

The number of genetic variants for a given genome can be enormous, and the effect of a genetic variant can be either neutral, favorable, or deleterious to the fitness and performance of an organism.

As used herein, the term “primary genetic variant” refers to a genetic variant having an effect as compared to the reference sequence or the wild-type sequence. A primary genetic variant may have a favorable or deleterious effect to the fitness and performance of an organism as compared to the reference sequence or the wild-type sequence. For example, in some embodiments, the primary genetic variant is a deleterious genetic variant having a deleterious or disease-causing effect as compared to the reference sequence. In some other embodiments, the primary genetic variant is a beneficial genetic variant having a beneficial or disease-preventing effect as compared to the reference sequence.

As used herein, the term “secondary genetic variant” refers to a genetic variant existing in addition to a primary genetic variant. A secondary genetic variant alone may or may not have an effect as compared to the reference sequence or the wild-type sequence. In some embodiments, a secondary genetic variant, when co-occurring with a primary genetic variant, can alter the effect of the primary genetic variant. In some embodiments, a secondary genetic variant can compensate for (e.g., counteract, offset, and/or oppose) the effect of the primary genetic variant. In some embodiments, the primary genetic variant is a deleterious genetic variant having a deleterious or disease-causing effect as compared to the reference sequence, and a secondary genetic variant can compensate for the deleterious or disease-causing effect of the primary genetic variant.

### Prediction of Compensatory Variants

With reference to FIG. 1A, the machine-learning model 120 is configured to predict the probabilities of one or more secondary genetic variants having a compensatory effect to the primary genetic variant, or the magnitudes of said effects.

Compensatory Genetic Variants

As used herein, a “compensatory” or “compensating” effect refers to a counteracting, offsetting, mitigating, and/or opposing effect. For example, relevant to a primary genetic variant, a “compensatory” or “compensating” secondary genetic variant would have a “compensatory effect” that counteracts, offsets, mitigates, and/or opposes the effect of the primary genetic variant. A compensatory secondary genetic variant may be within the same gene or gene product (e.g., polypeptide) as the primary genetic variant, i.e., a cis-acting compensatory genetic variant. A compensatory secondary genetic variant may be in a different gene or gene product (e.g., polypeptide) as the primary genetic variant, i.e., a trans-acting compensatory genetic variant. In some embodiments, the trans-acting compensatory genetic variant is within the same gene network as the primary genetic variant. Compensatory genetic variants are a manifestation of epistasis.

As used herein, the term “epistasis” (also known as “epistatic interaction” or “epistatic relationship”) refers to an interaction between variants of within or between genetic sequences, including, for example, genetic variants, where the presence of one genetic variant has an effect conditional on the presence of one or more additional genetic variants. Epistasis occurs both within and between molecules. Epistatic sequences may refer to alleles of a gene, genetic variants (e.g., mutations) of a gene, or sequences (e.g., genes, genetic variants) within a gene network or within a genome. Epistasis may be of various types, including, for example, dominant, recessive, complementary, compensatory, and polymeric interaction. A compensatory secondary genetic variant, for example, exhibits a compensatory epistatic interaction with a primary genetic variant. Various molecular mechanisms may contribute to epistasis, including, for example, the structure, stability, function, and interaction of nucleic acids and/or proteins, gene networks, metabolic networks, signaling pathways, etc. Due to its prevalence and multi-faceted nature, epistasis is an important factor contributing to the variation of many phenotypes, including human diseases, for which the identification of underlying epistasis is key to elucidating the genetic basis of complex diseases and leading to the development of treatments and therapeutics.

In some embodiments, the primary genetic variant is a deleterious genetic variant having a deleterious or disease-causing effect as compared to the reference sequence. Accordingly, a compensatory secondary genetic variant would counteract, offset, and/or oppose the deleterious or disease-causing effect of the primary genetic variant. For example, FIG. 1B illustrates the compensatory effect of a secondary genetic variant (e.g., a mutation) on maintaining proper and stable protein folding. The top row shows a wild-type (WT) gene model and the encoded properly folded protein, as well as the four potential mutation loci 1-4 on the WT gene model. The six gene models below the WT gene model show the various mutations (marked as “X”s) across mutation loci 1-4 on the WT. A triangle (A) at the site indicates the mutation, either alone or with other mutation(s) present in the same gene, does not affect proper and stable folding of the protein, i.e., having a non-pathogenic impact on the protein. A circle (O) at the site indicates the mutation, either alone or with other mutation(s) present in the same gene, prevents proper and stable folding of the protein, i.e., having pathogenic impact on the protein. The gene model on the bottom shows two mutations at locus 1 and locus 3 as a pair of compensatory mutations that lead to normal folding of the protein.

A compensatory secondary genetic variant may compensate for the primary genetic variant through various mechanisms. In some embodiments, the compensatory secondary genetic variant may change a conformational property of the protein, e.g., polar vs. non-polar, charged vs. no charge, positively charged (basic) vs. negatively charged (acidic), or hydrophobic vs. hydrophilic. In some embodiments, the compensatory secondary genetic variant may act in concert with the primary genetic variant (e.g., an active site mutation) by compensating for functional deficits caused by changes or mutations that affect binding in the active site.

In some other embodiments, the primary genetic variant is a beneficial genetic variant having a beneficial or disease-preventing effect as compared to the reference sequence. Accordingly, a compensatory secondary genetic variant would counteract, offset, and/or oppose the beneficial or disease-preventing effect of the primary genetic variant.

Experimental methods may be used to provide true compensatory genetic variants or validate predicted compensatory genetic variants. In some embodiments, the compensatory effect of a secondary genetic effect relative to a primary genetic variant is determined from a saturation mutagenesis analysis.

As used herein, the terms “site saturation mutagenesis (SSM),” “saturation mutagenesis,” and “site saturation,” refer to a random mutagenesis technique used in protein engineering, in which a single codon or set of codons is substituted with all possible amino acids at the position. Saturation mutagenesis is commonly achieved by site-directed mutagenesis PCR with a randomized codon in the primers (e.g., SeSaM) or by artificial gene synthesis, with a mixture of synthesis nucleotides used at the codons to be randomized. Variants of saturation mutagenesis are also known in the art, from paired site saturation (saturating two positions in every mutant in the library) to scanning site saturation (performing a site saturation at every site in the protein, resulting in a library of size that contains every possible point mutant of the protein). See more details in e.g., Chronopoulou, E. G., and Labrou, N. E., 2011. Site-saturation mutagenesis: A powerful tool for structure-based design of combinatorial mutation libraries. Current protocols in protein science, 63(1), pp. 26-6 and Shivange, A. V., Marienhagen, J., Mundhada, H., Schenk, A. and Schwaneberg, U., 2009. Advances in generating functional diversity for directed protein evolution. Current opinion in chemical biology, 13(1), pp. 19-25.

Predictive Modeling with Transfer Learning

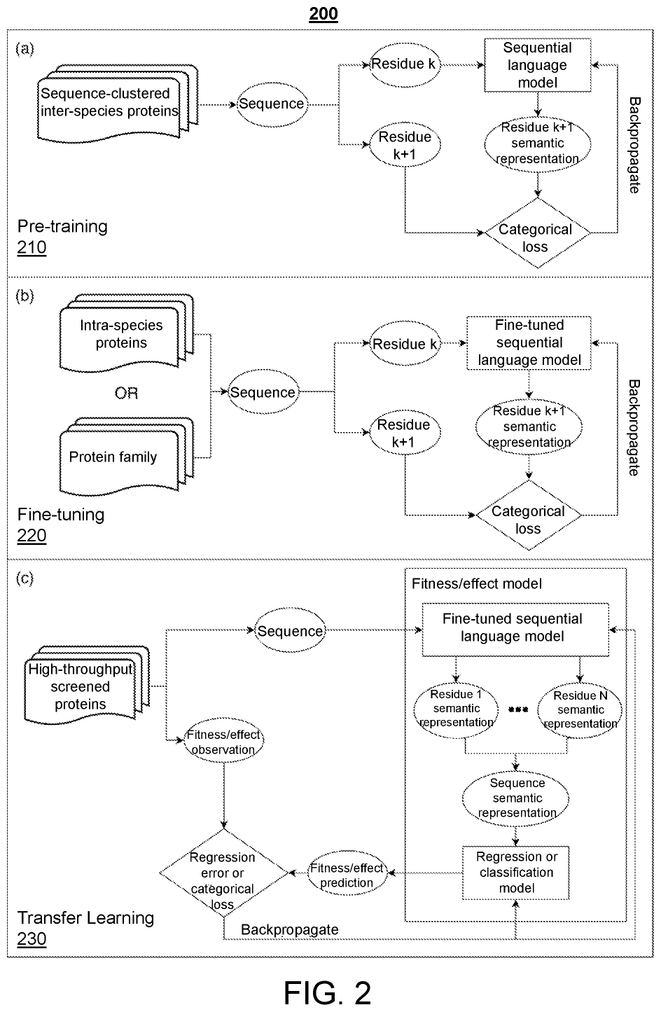

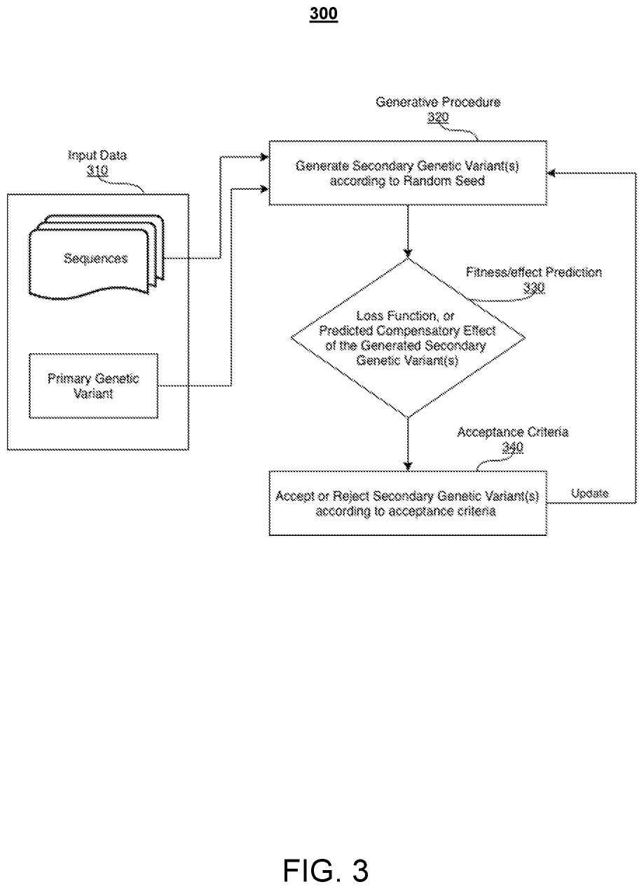

Various machine learning methods and modeling techniques may be used with the methods of the present disclosure. FIGS. 2 and 3 illustrate exemplary processes 200 and 300, respectively, for training the machine learning model (e.g., model 120) in accordance with some embodiments.

In some embodiments, the machine learning-based methods of the present disclosure use non-additive effects (e.g., epistasis or compensatory effect) to locate degenerate surfaces in the fitness landscape of genetic variants. This is especially useful for evaluating the off-target effects of various targeted procedures, such as genome editing and precision medicine.

Accordingly, FIG. 2 illustrates an exemplary training process using transfer learning to take advantage of neural network architectures optimized for language modeling.

With reference to FIG. 2, in some embodiments, the model is trained by: a) a pre-training task 210, comprising: 1) receiving a pre-training dataset comprising a plurality of batches of naturally occurring sequences; 2) inputting each batch of sequences into a language model, wherein the model is configured to output a pre-training set of semantic features; 3) automatically updating the language model after each batch; b) optionally, a fine-tuning task 220, comprising: 1) receiving a fine-tuning dataset comprising a plurality of batches of naturally occurring sequences, wherein the fine-tuning dataset is a subset of the pre-training dataset, or a set of sequences that are related to the pre-training dataset by common ancestry, homology, or multiple sequence alignment; 2) inputting each batch of sequences into the language model, wherein the model is configured to output a fine-tuning set of semantic features; and 3) automatically updating the language model after each batch; and c) a transfer learning task 230, comprising: 1) receiving a final training dataset comprising labeled sequences mapped to effects; and 2) training a neural network model based on the final training dataset, wherein the neural network model is configured to receive data corresponding to the pre-training set of semantic features and/or the fine-tuning set of semantic features, and output one or more effect scores.

As used herein, the term “language model,” which may refer to either a “sequential language model” or a “masked language model” refers to a machine learning method that interprets, predicts, and/or generates sequential data. At a high level, a sequential language model takes in a sequence of inputs, examines each element of the sequence, and predicts the next element of the sequence. Similarly, a masked language model takes in a sequence of inputs, a random subset of which have their ground truth masked or obscured from the perspective of the model, and predicts those masked elements. In some embodiments, the language model is a mathematical representation of the frequency and order with which specific monomeric units or gaps occur in a set of polymers, e.g., amino acid residues in a polypeptide sequence. The mathematical representation can include a probability of a given monomer occurring at a position in the sequence. In some embodiments, the language model predicts what specific monomer comes next in a sequence of different monomers—a process known as “next token prediction.” In some embodiments, the language model predicts what specific monomer should fill in a missing space in a sequence of different monomers—a process known as “masked token prediction.” A probability of a given monomer occurring at a position in the sequence model can be independent of other positions or can depend on the occupancy at any or all other positions in the sequence model. An example of a position independent model is a Hidden Markov Model. In some embodiments, the language model is configured to output a set of semantic features.

As used herein, the term “semantic feature” refers to a representation of how the elements relate to or connect with each other in the input sequence data. In some embodiments, the representation is mathematical or numerical. In some embodiments, the semantic features may be a human and/or machine interpretable representation of the state of the input sequence. The output semantic features may be presented in a vector or a matrix, and may be used as input for a downstream task, such as in transfer learning.

In some embodiments, the methods of the present disclosure utilize a language model to convert nucleotide or polypeptide sequences to numerical features. This encoding process is different from other processes, such as those that use the Fourier transform methods in digital signal processing. Without wishing to be bound by any theory, using a language model is postulated to contribute to the superior efficiency and effectiveness of the methods in the present disclosure.

As used herein, the term “transfer learning” refers to a machine learning method that stores knowledge learned from performing one task/solving one problem and transfers the learned knowledge to apply to a different but related task/problem. For example, a pre-trained model developed for a task may be used as the starting point for a model on a second task. In some embodiments of the present disclosure, the semantic representation learned from the language model in the pre-training task and/or the fine-tuning task may be transferred to use in the neural network model.

In some embodiments, the input data comprises a large, curated dataset of naturally occurring raw or aligned protein sequences that are evolutionarily sanctioned. Examples of such databases are the UniRef, UniParc, or Pfam databases.

In some embodiments, the dataset may be clustered by minimum sequence similarity in order to prevent overfitting. However, this reduces the resolution of sequence space sampled. This can be overcome by fine-tuning the model by training later on a particularly relevant cluster or set of clusters.

In some embodiments, the language model trains in a self-supervised manner on batches of raw amino acid sequences as input. Because the training strategy of this model is self-supervised, there is no need for any difficult or expensive preprocessing step.

In some embodiments, due to the evolutionary constraints imposed upon the input data, the internal state or parameterization of the model is obliged to approximate the distribution of sequential and evolutionarily-allowed runs of amino acids. However, under the assumption of independent and identically distributed training data, the approximation becomes increasingly accurate in the large data limit.

In some embodiments, the language model is rewarded by its ability to successfully predict the next or masked elements in the sequence, and/or penalized if otherwise.

In some embodiments, the model parameters of the language model are updated accordingly after each batch of input sequences.

In some embodiments, because the language model fits a probability distribution based only on the sequence space sampled in the dataset, the dependence of predictions on physical interactions with the environment is implicit. One can think of this type of model as a mean field theory with environmental degrees of freedom averaged over the full biologically-active range. The probability distribution can be made more specific to a particular environment through fine-tuning the language model on input sequences occurring naturally in this environment.

In some embodiments, the probability distribution has a parameterization defined by a learned set of semantic features, which together form a vector space. These semantic features can later be interrogated for pertinence to a particular downstream physical property of a protein.

In some embodiments, optionally, a second, smaller dataset (e.g., labeled dataset), mapping raw or aligned protein sequences to some desired physical property are used to fine-tune the language model. This fine-tuning dataset may be generated via a high-throughput screen. Depending on the physical property in question (e.g., protein stability), either an existing public dataset or an experimental protocol may be used. In some embodiments, the objective of this dataset is to probe the semantic vector space from the pre-training task in order to select features salient to the effect of interest.

In some embodiments, a labeled dataset is passed to a deep neural network comprising: 1) an upstream deep neural network equivalent to the language model with pre-trained weights; 2) a downstream randomly-initialized, shallow, and appropriately regularized neural network; and 3) an output layer with activation range equal to the range of the measured physical property (i.e., stability), wherein the output is referred to as an effect or fitness score.

In some embodiments, the output of the upstream neural network is a deep semantic representation vector for each nucleotide or amino acid in the sequence, which can be reduced to a sequence summary representation vector by applying an aggregation procedure to the position-specific representation vectors.

In some embodiments, over the course of training, the model learns a projection of the semantic vector space down to a lower-dimensional subspace of features salient to the physical property characterized by the training dataset.

In some embodiments, under the assumption that the probability distribution learned by the language model restricts to the distribution of the desired physical property, the upstream neural network can be held fixed during training. In this case, the downstream neural network is a simple map from semantic feature space to the active range of a specific physical property.

In some embodiments, wherein the above assumption does not hold, or if the distribution of the desired physical property is too fine-grained to be captured by the probability distribution of the language model, then the upstream neural network weights can be allowed to vary during training. This results in a deformation of the learned semantic space itself in order to capture more property-specific detail, leading to a more accurate projection down to the active range of the property.

Without wishing to be bound by any theory, some embodiments of the disclosed methods provide the superior ability to pre-compute a topographical map or fitness landscape of sequence space with contours representing surfaces of degenerate compensatory effect with respect to a given primary sequence variant. Non-limiting applications of this effect degeneracy map include: 1) allowing screening for higher-order mutational effect interactions; and 2) seeding new diversity within a species without affecting the biological pathways of the current generation such that proteins with altered sequences predicted by the degeneracy map lead to similar biological pathway outcomes or organismal phenotypes.

With reference to FIG. 2, the pre-training procedure 210 and fine-tuning procedure 220 of the model minimize the loss function (e.g., categorical loss) associated with next or masked sequence elements. During pre-training and fine-tuning, the model updates iteratively by back-propagating the loss to the parameters of the model and optimizes semantic representation of the sequences.

With reference to FIG. 2, the transfer learning procedure 230 of the model minimizes the loss function (e.g., regression error or categorical loss) associated with the prediction of the compensatory effect of secondary genetic variants. During transfer learning, the model updates iteratively by back-propagating the loss to the parameters of the model and optimizes the output effect scores of the compensatory secondary genetic variants. In some embodiments, all parameters of the model are updated. In some embodiments, only the parameters of the final few layers of the neural network are updated, with the rest of the layers held fixed.

Generative Modeling

FIG. 3 illustrates an exemplary training process using generative modeling.

As used herein, a “generative model” or “generative procedure” refers to a model, such as a machine learning model that is trained using a set of data, which as a result of being trained, can generate new targets that follow the probability distribution of the training set. A generative model can be used to implement an unsupervised learning system. A generative model can generate the observed values used to train it and variables that can be modeled based on their fit to the probability distribution of the training set.

Accordingly, in some embodiments, the machine learning-based methods in the present disclosure utilizes a generative model to identify compensatory genetic variants, which is useful for reducing or eliminating false positive candidates (e.g., non-functional or ineffective genetic variants) for use in targeted procedures, such as genome editing and precision medicine.

With reference to FIG. 3, in some other embodiments, the model is trained by: a) receiving a training dataset of sequences 310, comprising a training reference sequence and a training primary genetic variant, wherein the training primary genetic variant has an effect on the reference sequence with respect to a metric of interest; b) inputting the training dataset into a generative procedure configured to generate one or more training secondary genetic variants according to a random seed 320; c) calculating a loss function, wherein the loss function 330 maps the combined effect of the primary and secondary genetic variants and the effect of the reference sequence onto a quantitative error score; d) accepting or rejecting the one or more training secondary genetic variants according to one or more pre-determined acceptance criteria on the loss function 340; e) updating the generative procedure by incorporating the accepted one or more training secondary genetic variants in a new round of additional training secondary genetic variants; and f) repeating steps b) to e) until the loss converges to a minimum.

The training procedure 300 of the model minimizes the loss function (e.g., a binary loss or a distance metric) associated with the prediction of the compensatory effect of secondary genetic variants. During training, the model updates iteratively by back-propagating the loss to the parameters of the model and optimizes the output effect scores of the compensatory secondary genetic variants.

### Output and Assessment

Referring back to FIG. 1A, the output 130 is one or more effect scores corresponding to the probabilities of one or more secondary genetic variants having a compensatory effect to the primary genetic variant, or the magnitudes of said effects, as predicted by the machine-learning model 120.

As used herein, the terms “effect score” and “fitness score” refer to a representation of the effect or fitness of a secondary genetic variant relative to the primary genetic variant, in the context of a reference or wild-type sequence. The representation may be interpretable to humans and/or machines. In some embodiments, the representation is a numerical representation.

In some embodiments, a genetic variant may not produce a detectable, functional effect. For example, a genetic variant may be a single nucleotide substitution when the change in the DNA base sequence results in a new codon still coding for the same amino acid, e.g., a sense mutation. In some other embodiments, a genetic variant may produce a detectable or functional effect such as, for example, a decrease in function of a gene product, ablation of function in a gene product, and/or a new function in a gene product. In some embodiments, the effect is an effect at a molecular level, a cellular level, a sub-organismal level, or an organismal level. In some embodiments, the effect is an effect affecting an endophenotype selected from a group consisting of messenger RNA (mRNA) abundance, gene transcript splicing ratio, protein abundance, micro RNA (miRNA) or small RNA (siRNA) abundance, translational efficiency, ribosome occupancy, protein modification, metabolite abundance, allele specific expression (ASE), and visual feature measured at the sub-organismal level. In some embodiments, the effect is an effect affecting a protein property. In some embodiments, the effect is an effect affecting protein structure, protein conformation, protein molecular or cellular function, protein stability, enzymatic affinity, or enzymatic efficiency. In some embodiments, the effect is a collection of effects characterizing the state of a protein. In some embodiments, the effect is an effect affecting fitness or performance of an organism. In some embodiments, the effect is interpretable to humans and/or machines.

Because the number of genetic variants for a given genome can be enormous, in some embodiments, the output effect scores are further assessed.

In some embodiments, the method further comprises selecting one or more secondary genetic variants based on the effect scores.

In some embodiments, the method further comprises prioritizing or ranking one or more secondary genetic variants based on the effect scores.

In some embodiments, the method further comprises evaluating epistasis of one or more secondary genetic variants based on the effect scores.

Methods and techniques to study and investigate epistasis are known in the art. Accordingly, a skilled artisan would be able to choose suitable methods and techniques to evaluate epistasis of one or more predicted secondary genetic variants using methods of the present disclosure. Further descriptions may be referred to, for example, Cordell, H. J., 2002. Epistasis: what it means, what it doesn't mean, and statistical methods to detect it in humans. Human molecular genetics, 11(20), pp. 2463-2468; Moore, J. H., 2003. The ubiquitous nature of epistasis in determining susceptibility to common human diseases. Human heredity, 56(1-3), pp. 73-82; Moore, J. H. and Williams, S. M., 2009. Epistasis and its implications for personal genetics. The American Journal of Human Genetics, 85(3), pp. 309-320; Lehner, B., 2011. Molecular mechanisms of epistasis within and between genes. Trends in Genetics, 27(8), pp. 323-331; Mackay, T. F., 2014. Epistasis and quantitative traits: using model organisms to study gene-gene interactions. Nature Reviews Genetics, 15(1), pp. 22-33; and Hartman, E. C. and Tullman-Ercek, D., 2019. Learning from protein fitness landscapes: a review of mutability, epistasis, and evolution. Current Opinion in Systems Biology, 14, pp. 25-31. In contrast to the methods known in the art which predict the existence of epistasis between two or more variants, the methods described herein predict the impact on endophenotypes or organismal fitness of pairwise or higher-order combinations of genetic variants. One important difference and advantage of the present invention over the art is that these interacting genetic variants and their combined effect can be predicted using the methods disclosed herein, regardless if they are observed or are not observed in nature either because one or more of the genetic variants are not observed to occur in nature, or because the combination of genetic variants does not occur in nature.

In some embodiments that may be combined with any of the preceding embodiments, the method further comprises: a) altering one or more of the secondary genetic variants in the genome of an organism; b) identifying an impact of the alteration on an endophenotype, wherein the endophenotype is a quantifiable phenotype at a sub-organismal level that can be measured by a biochemical, gene expression, or protein level assay; and c) updating the model using the identified endophenotypic impact.

As used herein, the term “endophenotype” refers to a quantifiable phenotype at the sub-organismal level that can be measured by a biochemical, gene expression, protein level assay, or visual feature measured at the sub-organismal level. In some embodiments, the endophenotype is an intermediate quantitative phenotype that is biologically relevant to, associated with, or predicative of a phenotype at the organism level, such as yield performance or overall fitness. Endophenotypes can be readily measured in cells, tissue, or young organisms that serve as a proxy to determine quickly which genetic variants are more likely to have an impact on a terminal phenotype, such as yield performance or overall fitness. Examples of endophenotypes include, but are not limited to, messenger RNA (mRNA) abundance, gene transcript splicing ratio, protein abundance, micro RNA (miRNA) or small RNA (siRNA) abundance, translational efficiency, ribosome occupancy, protein modification, metabolite abundance, and allele specific expression (ASE). Endophenotypes may be associated with a genetic variant that is physically proximal or proximal within a gene network.

Methods and techniques for measuring quantifiable phenotypes by biochemical, gene expression, or protein level assays are known in the art. A skilled artisan would be able to determine the suitable methods and techniques based on their specific circumstances. Non-limiting examples of biochemical assays include the refractive index spectroscopy (RI), ultra-violet spectroscopy (UV), fluorescence analysis, radiochemical analysis, near-infrared spectroscopy (near-IR), nuclear magnetic resonance spectroscopy (NMR), light scattering analysis (LS), mass spectrometry, pyrolysis mass spectrometry, nephelometry, dispersive Raman spectroscopy, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, NMR and IR detection (See e.g., WO 04/056456 and WO 04/088309). Non-limiting examples of methods for quantifying mRNA expression include northern blotting and in situ hybridization (Parker & Barnes, Methods in Molecular Biology 106:247 283 (1999)); RNAse protection assays (Hod, Biotechniques 13:852 854 (1992)), and PCR-based methods, such as reverse transcription polymerase chain reaction (RT-PCR) (Weis et al., Trends in Genetics 8:263 264 (1992)). Expression levels of purified protein in solution can be determined by physical methods, e.g. photometry. Methods of determining the expression level of a particular protein in a mixture rely on specific binding, e.g., of antibodies. Specific detection and quantitation methods exploiting the specificity of antibodies comprise immunohistochemistry (in situ) and surface plasmon resonance. Western blotting combines separation of a mixture of proteins by electrophoresis and specific detection with antibodies. Other means of determining protein expression data include two-dimensional gel-electrophoresis, preferably in combination with mass spectrometry. With the advent of microarray technology, measurement of protein expression levels in array format became increasingly widespread. Protein arrays for determining protein expression data exploit interactions such as protein-antibody, protein-protein, protein-ligand, protein-drug and protein-small molecule interactions or any combination thereof. Protein expression data reflect, in addition to regulation at the transcriptional level, regulation at the translational level as well as the average lifetime of a protein prior to degradation.

In some embodiments, the compensatory genetic variants by the methods of the present disclosure may be further assessed, weighted, or prioritized by a statistical model based on one or more criteria. Examples of the criteria include, but are not limited to, evolutionary conservation (See e.g., Chun and Fay (2009) Genome Res. 19: 1553-1561 and Rodgers-Melnick et al. (2015) PNAS 112: 3823-3828), functional impact of amino acid change (See e.g., Ng et al. (2003) NAR 31:3812-3814 and Adzhubei et al. (2010) Nat Methods 7:248-249), functional impact of protein conformation and/or stability (See e.g., Rosetta, a computational protein design platform from Cyrus Bio Inc.), adjacency to selective sweep regions (See e.g., Hufford et al. (2012) Nat gen 44: 808-813), and outlier status of an endophenotype (See e.g., Zhao et al. (2016) AJHG 98, 299-309).

### Use in Synthetic Biology

Another aspect of the present disclosure relates to using the disclosed methods in the field of synthetic biology. In one embodiment, provided herein is a method for designing a molecule with a desired effect, comprising: a) receiving a dataset of sequences from an input device, wherein the dataset of sequences comprises a reference sequence, a primary genetic variant of the reference sequence, and one or more secondary genetic variants in the reference sequence; b) automatically inputting the dataset of sequences to a trained machine-learning model to obtain one or more effect scores, wherein the model is configured to output one or more effect scores corresponding to the probabilities of one or more secondary genetic variants having a compensatory effect to the primary genetic variant, or the magnitudes of said effects; c) displaying the predicted effect scores on a display device; and d) designing a molecule based on the effect scores.

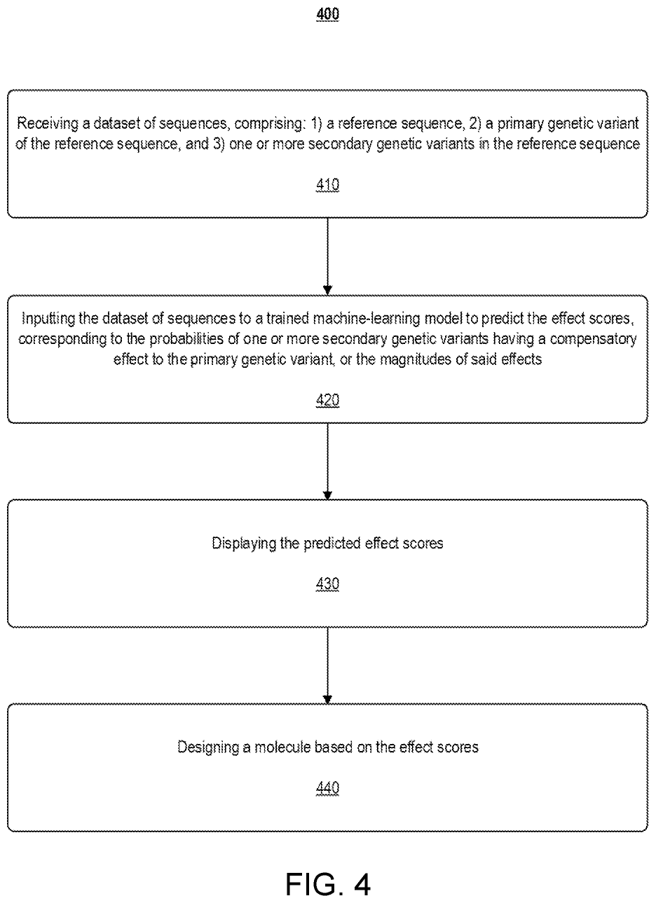

FIG. 4 illustrates an example of such a method 400 in accordance with some embodiments. In some embodiments, after the step 440, the method further comprises synthesizing the designed molecule.

As used herein, the term “synthetic biology” refers to the design and construction of new biological entities such as enzymes, genetic circuits, and cells or the redesign of existing biological systems. Synthetic biology builds on the advances in molecular, cell, and systems biology and seeks to transform biology in the same way that synthesis transformed chemistry and integrated circuit design transformed computing. Detailed description may be referred to e.g., Benner, S. A. and Sismour, A. M., 2005. Synthetic biology. Nature Reviews Genetics, 6(7), pp. 533-543; and Ruder, W. C., Lu, T. and Collins, J. J., 2011. Synthetic biology moving into the clinic. Science, 333(6047), pp. 1248-1252.

The ability to design and synthesize bio-molecules de novo is an important part of synthetic biology. Therefore, the machine learning-based methods of the present disclosure are useful for computationally designing and synthesizing molecules with desired characteristics, such as an engineered protein with increased stability.

The effect of higher order variant combinations on protein stability can be predicted by free energy calculations such as those performed by Rosetta (Cyrus biotechnology). However, the use of Rosetta is limited for the following reasons, which are solved when predicting protein properties directly from sequences using methods of the present disclosure.

Specifically, computational protein modeling software, such as Rosetta, which rely on free energy calculations to determine the physical properties of the molecule are limited by: 1) laborious and expensive preprocessing of input data (e.g., crystal structure), 2) highly-constrained environmental assumptions, and 3) high computational complexity.

Crystal structures are required for Rosetta free energy stability predictions. This information is available for smaller than 1% of all known proteins and almost no proteins outside of humans and a few model species.

Prediction of stability via free energy calculations is computationally expensive, and inference of coding sequence mutation effects in tens of thousands of proteins and hundreds of thousands of their mutated variants may be prohibitive in both cost and time.

Free energy-based stability calculations also require a user to select a radius in the protein in which amino acids will be repacked around a particular mutated site.

Similarly, when using free energy-based calculations to predict protein stability, cytosolic pH and mesophilic conditions are assumed, which limits relevance for applications in synthetic biology, extracellular proteins, or in extremophiles.

Some additional limitations of proteins accessible to Rosetta include: only proteins in the PDB database; no intrinsically disordered proteins; no structural proteins; only crystallizable proteins; only mesophilic conditions.

Accordingly, the machine learning-based methods of the present disclosure are useful in aiding the design and synthesis of molecules with various desired effects, e.g., in protein engineering. In some embodiments, the machine learning-based methods of the present disclosure predict the likelihood of a genetic variant having a compensatory effect, or magnitude thereof. For instance, the methods of the disclosure can indicate the probability or magnitude of a change in effect of epistatic mutations, e.g., switching between neutral, deleterious, and beneficial.

In some embodiments, the machine learning-based methods of the present disclosure identify specific epistatic interactions in genetic variants, including, for example, dominant, recessive, complementary, compensatory, or polymeric interaction.

In some embodiments, the effect of the designed molecule is stability, solubility, affinity, biological activity, bioavailability, a chemical property, a physical property, or a structural property.

Various types of molecules may be designed using the methods described herein. In some embodiments, the designed molecule is a DNA molecule, an RNA molecule, or a protein molecule. In some embodiments, the designed molecule is a single stranded DNA (ssDNA) or a double stranded DNA (dsDNA). In some embodiments, the designed molecule is a messenger RNA (mRNA), a transfer RNA (tRNA), a ribosomal RNA (rRNA), a small RNA (sRNA), or a guide RNA (gRNA). In some embodiments, the designed molecule is an antibody, a contractile protein, an enzyme, a hormonal protein, a structural protein, a storage protein, or a transport protein. In some embodiments, the designed molecule is a viral molecule, a bacterial molecule, an algal molecule, a fungal molecule, a plant molecule, an animal molecule, or a human molecule. In some embodiments, the designed molecule is a virus protein. In some embodiments, the virus protein is a protein from a coronavirus. In some embodiments, the coronavirus is a severe acute respiratory syndrome coronavirus 2 (SARS-CoV-2) that is the causal agent for the infectious disease coronavirus disease 2019 (COVID-19).

### Use in Personalized Medicine

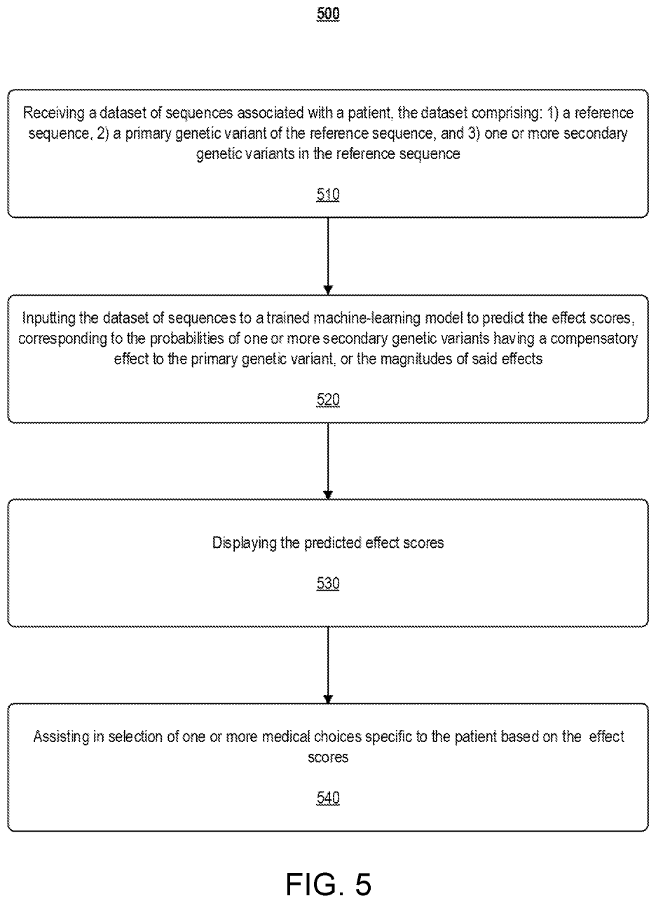

In yet another aspect, provided herein is a method for providing personalized and probabilistic information for a patient, comprising: a) receiving a dataset of sequences associated with a patient from an input device, wherein the dataset of sequences comprises a reference sequence, a primary genetic variant of the reference sequence, and one or more secondary genetic variants in the reference sequence; b) automatically inputting the dataset of sequences to a trained machine-learning model to obtain one or more effect scores, wherein the model is configured to output one or more effect scores corresponding to the probabilities of one or more secondary genetic variants having a compensatory effect to the primary genetic variant, or the magnitudes of said effects; c) displaying the predicted effect scores on a display device; and d) causing selection of one or more medical choices specific to the patient based on the effect scores, as illustrated by the exemplary process 500 in FIG. 5. In some embodiments, the method further comprises recommending an intervention or a therapeutic agent based upon the effect score.

In some embodiments, the one or more medical choices are selected from the group consisting of prognosis, diagnosis, treatment, intervention, and prevention.

In some embodiments, provided herein is a method of treatment, comprising: a) receiving a dataset of sequences associated with a patient from an input device, wherein the dataset of sequences comprises a reference sequence, a primary genetic variant of the reference sequence, and one or more secondary genetic variants in the reference sequence; b) automatically inputting the dataset of sequences to a trained machine-learning model to obtain one or more effect scores, wherein the model is configured to output one or more effect scores corresponding to the probabilities of one or more secondary genetic variants having a compensatory effect to the primary genetic variant, or the magnitudes of said effects; c) displaying the predicted effect scores on a display device; d) assisting in selection of one or more medical treatments specific to the patient based on the effect scores; and e) administering the one or more medical treatments to the patient.

As used herein, the terms “personalized medicine,” “individualized medicine,” and “precision medicine” refer to the tailoring of medical treatment to the individual characteristics of each patient, based on the patient's unique molecular and genetic profile that make the patient predisposed or susceptible to certain diseases. Personalized medicine is increasing the ability to predict which medical treatments will likely be safe and effective for each patient, and which ones will likely not be.

Compensatory genetic variants are important factors to consider in a patient's genetic makeup. In human populations it is observed that 10% of identified deleterious sites are locally complemented by another mutation (Kondrashov et al. 2002) based on disease driving mutations being re-observed in related mammals in non-disease presenting individuals but only in the presence of a second, third or fourth, etc., mutation. This suggests that if prioritizing variants that are derived alleles on a site-basis, 10% of mutations will be misclassified as functionally deleterious despite local compensation by another mutation (or mutations) that abrogates the original mutation's effect. Misclassification leads to prioritization of mutations that are in fact benign. In the case of a pair of compensating mutations, this occurs because the mutational target space for a population to develop and select for a compensating mutation at another nearby site is often larger than the chance of having a mutation that reverts the original mutation back to its ancestral state. A lack of accurate assessment of the total functional tumor mutation burden (TMB) reduces accuracy in predicting response to treatment. Specifically, TMB assessment predicts immunotherapy response, which guides patient and clinician decision-making about pursuing treatment (Balar et al. 2017). Calculating gross TMB without regard to which sites are functional and which sites may in fact be compensating for each other to restore wild-type function reduces the accuracy of this metric.

Accordingly, the methods of the present disclosure may be used to assess: 1) disease risk in carrier screening, and 2) genetic profiling of cancer tumors to guide treatment, among other applications of personalized medicine.

In some embodiments, the attribute associated with the patient is selected from the group consisting of genetic profile, predisposition or response to a disease, and response to a treatment.

In some embodiments, the genetic profile is from one or more cancer tumors of the patient.

The methods of the present disclosure may be used with various diseases. In some embodiments, the disease is selected from the group consisting of cancer, obesity, hypertension, a cardiovascular disease, an infectious disease, an autoimmune disease, a genetic disease, a liver disease, insulin resistance, Crohn's disease, dementia, Alzheimer's disease, cerebral infarction, hemophilia, viral hepatitis, sickle cell disease, multiple sclerosis, and muscular dystrophy.

Various treatments may be used with the methods of the disclosure. In some embodiments, the treatment is selected from the group consisting of drug administration, chemotherapy, radiation therapy, immunotherapy, and gene therapy.

By way of example, given a panel of potential drugs prescribed given mutations in different genes/proteins, if multiple of these genes/proteins have mutations but only some of them are producing functional protein, one can use the methods of the present disclosure to efficiently and effectively select drugs that target genes/proteins that are still likely to be functional and stable (e.g., by having compensatory secondary genetic variants), instead of the knocked-out genes/proteins given that those more likely no longer contain active cancer driving mutations.

### Use in Predicting Treatment Response

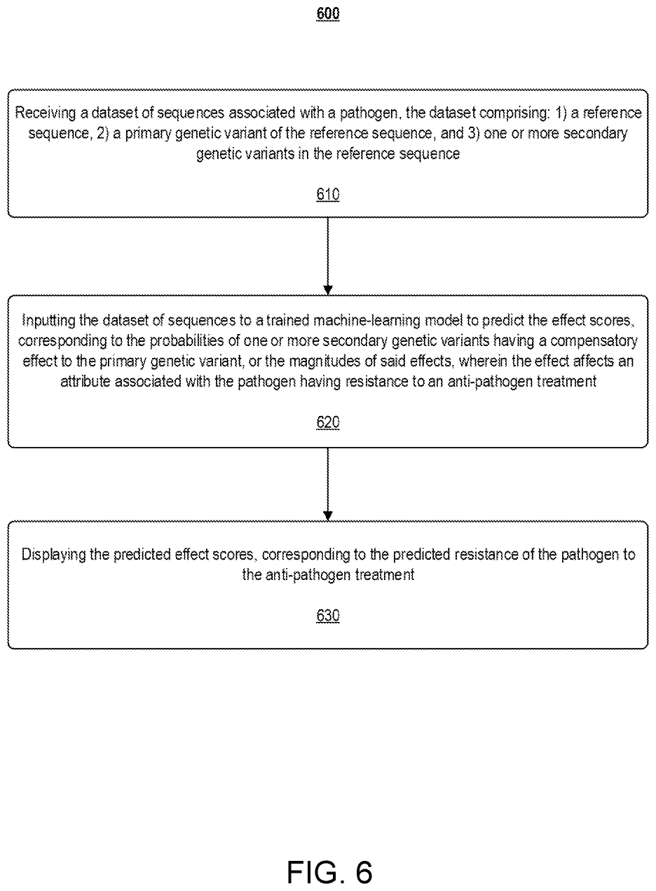

In still another aspect, provided herein is a method for predicting resistance of a pathogen to an anti-pathogen treatment, comprising: a) receiving a dataset of sequences associated with a pathogen from an input device, wherein the dataset of sequences comprises a reference sequence, a primary genetic variant of the reference sequence, and one or more secondary genetic variants in the reference sequence; b) automatically inputting the dataset of sequences to a trained machine-learning model to obtain one or more effect scores, wherein the model is configured to output one or more effect scores corresponding to the probabilities of one or more secondary genetic variants having a compensatory effect to the primary genetic variant, or the magnitudes of said effects, wherein the effect affects an attribute associated with the pathogen having resistance to an anti-pathogen treatment; and c) displaying the predicted effect scores on a display device, corresponding to the predicted resistance of the pathogen to the anti-pathogen treatment, as illustrated by the exemplary process 600 in FIG. 6.

In some embodiments, the method further comprises administering one or more treatments according to the predicted resistance. In some embodiments, the one or more treatments comprise an alternative treatment that is different from the treatment predicted to be resisted by the pathogen without considering pairwise or higher-order mutational interactions in the genome of the pathogen. In some embodiments, for instance when the primary genetic variant confers resistance and the identified compensatory secondary genetic variants alleviate or eliminate the resistance, the one or more treatments comprise a treatment typical for the pathogen that would have otherwise not be recommended based on presence of the primary genetic variant alone.

In some embodiments, provided herein is a method of treatment, comprising: a) receiving a dataset of sequences associated with a pathogen from an input device, wherein the dataset of sequences comprises a reference sequence, a primary genetic variant of the reference sequence, and one or more secondary genetic variants in the reference sequence; b) automatically inputting the dataset of sequences to a trained machine-learning model to obtain one or more effect scores, wherein the model is configured to output one or more effect scores corresponding to the probabilities of one or more secondary genetic variants having a compensatory effect to the primary genetic variant, or the magnitudes of said effects, wherein the effect affects an attribute associated with the pathogen having resistance to an anti-pathogen treatment; c) displaying the predicted effect scores on a display device, corresponding to the predicted resistance of the pathogen to the anti-pathogen treatment; and d) administering one or more treatments according to the predicted resistance of the pathogen. In some embodiments, the one or more treatments comprise an alternative treatment that is different from the treatment having predicted resistance by the pathogen using less sophisticated methods.

The ability to predict which mutations in a viral or bacterial genome allow viruses and bacteria to gain resistance to antivirals or antibiotics also enables more accurate prescription of treatment at reduced cost and with reduced risk of creating additional antibiotic resistance. However, the impact of each successive mutation that modifies the susceptibility of the organism to the antimicrobial must be conditioned on the presence of the other mutations that have occurred. For example, in the case of Neisseria gonorrhea, most infections are treatable with quinolone antibiotics such as ciprofloxacin, but a minority of infections can only be treated with the newest third generation cephalosporin class of antibiotics like ceftriaxone (Tapsall 2005). If one were to only consider single-site mutations, a given variant of the infection may be deemed resistant to ciprofloxacin, when in reality it also contains a secondary mutation that increases its susceptibility. In this case, a doctor may be likely to prescribe the alternative treatment, and unnecessarily increase selective pressure on resistance of ceftriaxone.

The present disclosure may be used for various pathogens. In some embodiments, the pathogen is a virus, a prion, a viroid, a bacterium, a fungus, a protozoan, or a parasite.

In some embodiments, the attribute associated with the pathogen is selected from the group comprising nucleic acid replication, DNA integration into a host genome, gene expression, protein synthesis, metabolism, cell membrane synthesis, cell wall synthesis, and peptidoglycan biosynthesis.

Various treatments may be used with the methods of the disclosure. In some embodiments, the anti-pathogen treatment is administering a drug selected from the group consisting of an antiviral, an antibacterial, an antibiotic, an antifungal, an antiparasitic, and a pesticide.

In some embodiments, the pathogen is Neisseria gonorrhea and the anti-pathogen treatment is administration of ciprofloxacin or ceftriaxone.

### Use in Breeding and Genetic Engineering

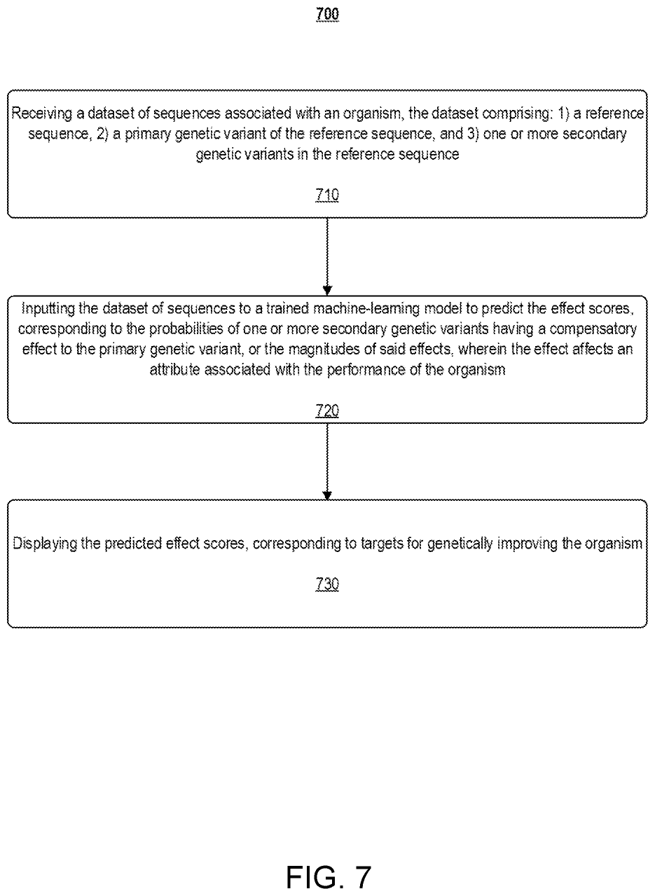

In still another aspect, provided herein is a method for identifying targets for genetically improving a trait in an organism, comprising: a) receiving a dataset of sequences associated with an organism from an input device, wherein the dataset of sequences comprises a reference sequence, a primary genetic variant of the reference sequence, and one or more secondary genetic variants in the reference sequence; b) automatically inputting the dataset of sequences to a trained machine-learning model to obtain one or more effect scores, wherein the model is configured to output one or more effect scores corresponding to the probabilities of one or more secondary genetic variants having a compensatory effect to the primary genetic variant, or the magnitudes of said effects, and wherein the effect affects an attribute associated with the trait of the organism; and c) displaying the predicted effect scores on a display device, corresponding to the targets for genetically improving the organism, as illustrated by the exemplary process 700 in FIG. 7.

In some embodiments, the method further comprises selecting one or more of the targets for genetic improvement of the organism.

In some embodiments, the method further comprises selecting an organism having the improved trait.

In some embodiments, provided herein is a method for genetically improving a trait in an organism, comprising: a) receiving a dataset of sequences associated with an organism from an input device, wherein the dataset of sequences comprises a reference sequence, a primary genetic variant of the reference sequence, and one or more secondary genetic variants in the reference sequence; b) automatically inputting the dataset of sequences to a trained machine-learning model to obtain one or more effect scores, wherein the model is configured to output one or more effect scores corresponding to the probabilities of one or more secondary genetic variants having a compensatory effect to the primary genetic variant, or the magnitudes of said effects, and wherein the effect affects an attribute associated with a trait of the organism; c) displaying the predicted effect scores on a display device, corresponding to the targets for genetically improving the organism; and d) altering the predicted targets to genetically improve the trait in the organism.

The targets identified from the methods of the present invention may be used for genetic improvement in agricultural organisms. With reference to FIG. 7, this step of genetic improvement of an organism may be carried out after step 730. Various methods and techniques of genetic improvement are known in the art and may be used in the present invention. For instance, genetic improvement may be achieved by conventional breeding, or with the help of biotechnology, such as marker assisted selection (MAS) or genetic engineering.

Molecular markers can be used during the breeding process for the selection of agriculturally important traits. For example, markers closely linked to the compensatory genetic variants identified from the methods of the present disclosure can be used to select individuals that contain the alleles of interest during a breeding program. The use of molecular markers in the selection process is often called genetic marker-enhanced selection or MAS.

Genetic engineering tools such as transgenic and genome-editing techniques may also be used for variety development and breeding. See, e.g., Moose, Stephen P., and Rita H. Mumm. “Molecular plant breeding as the foundation for 21st century crop improvement.” Plant physiology 147.3 (2008): 969-977, and Chen, Kunling, et al. “CRISPR/Cas genome editing and precision plant breeding in agriculture.” Annual review of plant biology 70 (2019): 667-697.

Additional non-limiting examples of plant variety development and breeding methods that may be used include, without limitation, those found in Principles of Plant Breeding, John Wiley and Son, pp. 115-161 (1960); Allard (1960); Simmonds (1979); Sneep, et al. (1979); Fehr (1987); and “Carrots and Related Vegetable Umbelliferae”, Rubatzky, V. E., et al. (1999). For further details of methods and techniques in animal variety development and breeding, see, e.g., Misztal I. (2013) Animal Breeding and Genetics, Introduction. In: Christou P., Savin R., Costa Pierce B. A., Misztal I., Whitelaw C. B. A. (eds) Sustainable Food Production. Springer, New York, NY.

In some embodiments, the genetic improvement is achieved by conventional breeding methods, such as selection.

In some embodiments, the genetic improvement is achieved by a transgenic technology or a genome editing technology.

In some embodiments, the genome editing technology is a base editing technology using a DNA base editor or an RNA base editor.

In some embodiments, the genome editing is achieved by a clustered regularly interspersed short palindromic repeats (CRISPR) system, a transcription activator-like effector nuclease (TALEN) system, or a zinc finger nuclease (ZFN) system.

In some embodiments, the genome editing is achieved by coupling with a recombination system.

In some embodiments, the recombination system is a lambda phage derived recombination (lambda Red) system.

It is contemplated that the methods described herein may be used in any suitable agricultural organisms. In some embodiments, the organism is selected from the group consisting of maize, soybean, wheat, sorghum, barley, oats, rice, millet, canola, cotton, cassava, cowpea, safflower, sesame, tobacco, flax, sunflower, a grain crop, a vegetable crop, an oil crop, a forage crop, an industrial crop, a woody crop, and a biomass crop. In some other embodiments, the organism is selected from the group consisting of cattle, sheep, pigs, goats, horses, mice, rats, rabbits, cats, and dogs.

In some embodiments, the organism is maize, wheat, barley, oat, rice, soybean, oil palm, safflower, sesame, tobacco, flax, cotton, sunflower, pearl millet, foxtail millet, sorghum, canola, cannabis, a vegetable crop, a forage crop, an industrial crop, a woody crop, or a biomass crop.

In some embodiments, the trait is yield, overall fitness, biomass, photosynthetic efficiency, nutrient use efficiency, heat tolerance, drought tolerance, herbicide tolerance, or disease resistance.

In some embodiments, the organism is cattle, sheep, goat, horse, pig, chicken, duck, goose, rabbit, or fish.

In some embodiments, the trait is growth rate, feed use efficiency, meat yield, meat quality, milk yield, milk quality, egg yield, egg quality, wool yield, or wool quality.

In some embodiments, provided herein is an organism genetically improved by the method of any of the preceding embodiments.

### Use in Genome/Base Editing

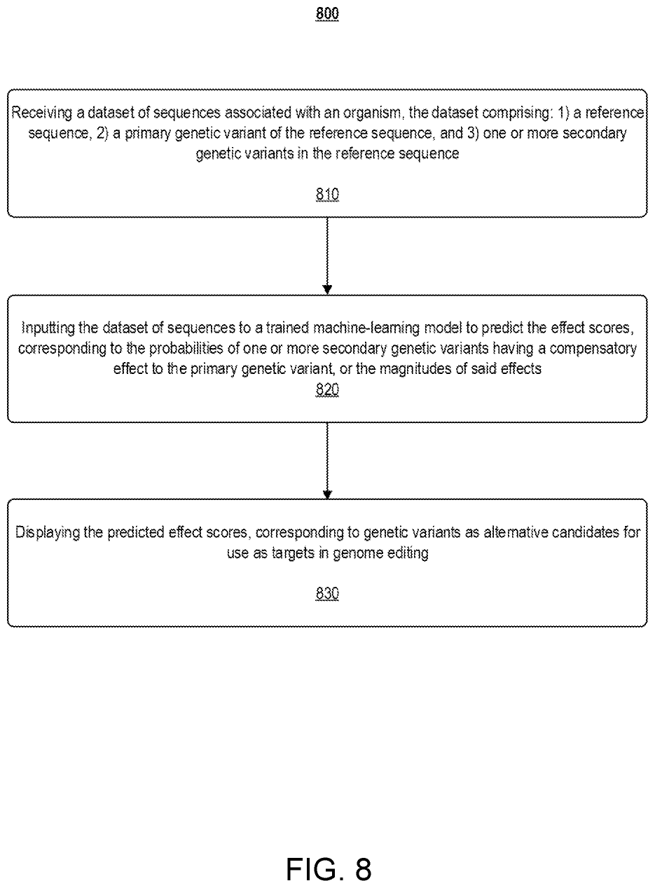

In still another aspect, provided herein is a method for identifying genetic variants as alternative candidates for use as targets that are more easily accessible by a transgenic technology or a genome editing technology, comprising: a) receiving a dataset of sequences associated with an organism from an input device, wherein the dataset of sequences comprises a reference sequence, a primary genetic variant of the reference sequence, and two or more secondary genetic variants in the reference sequence; b) automatically inputting the dataset of sequences to a trained machine-learning model to obtain one or more effect scores, wherein the model is configured to output one or more effect scores corresponding to the probabilities of one or more secondary genetic variants having a compensatory effect to the primary genetic variant, or the magnitudes of said effects; and c) displaying the predicted effect scores on a display device, corresponding to the genetic variants as alternative candidates for use as targets in genome editing, as illustrated by the exemplary process 800 in FIG. 8.

In some embodiments, the method further comprises producing the genetic variants identified as alternative candidate targets in genome editing.

In some embodiments, provided herein is a method for identifying genetic variants as alternative candidates for use as targets in genome editing, comprising: a) receiving a dataset of sequences associated with an organism from an input device, wherein the dataset of sequences comprises a reference sequence, a primary genetic variant of the reference sequence, and two or more secondary genetic variants in the reference sequence; b) automatically inputting the dataset of sequences to a trained machine-learning model to obtain one or more effect scores, wherein the model is configured to output one or more effect scores corresponding to the probabilities of one or more secondary genetic variants having a compensatory effect to the primary genetic variant, or the magnitudes of said effects; c) displaying the predicted effect scores on a display device, corresponding to the genetic variants as alternative candidates for use as targets in genome editing; and d) producing the genetic variants identified as alternative candidate targets in genome editing.

Targeted editing of nucleic acid sequences, for example, the targeted cleavage or the targeted introduction of a specific modification into genomic DNA, is a highly promising approach for the study of gene function and also has the potential to provide new therapies for human genetic diseases (Humbert et al., Crit Rev Biochem Mol (2012) 47(3):264-81. PMID: 22530743). Many genetic disorders have been identified as having specific nucleotide changes underlying the disorder (for example, a C to T change in a specific codon of a gene associated with a disease; Cargill et al., Nat Genet (1999) 22(3):231-8. PMID: 10391209).

As used herein, the terms “genome editing,” “gene editing,” and “targeted editing” refer to the process of altering the target genomic DNA sequence by inserting, replacing, or removing one or more nucleotides. Genome editing may be accomplished by using nucleases, which create specific double-strand breaks (DSBs) at desired locations in the genome, and harness the cell's endogenous mechanisms to repair the induced break by homology-directed repair (HDR) (e.g., homologous recombination) or by non-homologous end joining (NHEJ). Any suitable nuclease may be introduced into a cell to induce genome editing of a target DNA sequence including, but not limited to, clustered regularly interspersed short palindromic repeats (CRISPR)-associated protein (Cas, e.g., Cas9 and Cas12a) nucleases, zinc finger nucleases (ZFNs, e.g., FokI), transcription activator-like effector nucleases (TALENs, e.g., TALEs), meganucleases, and variants thereof (Shukla et al. (2009) Nature 459: 437-441; Townsend et al. (2009) Nature 459: 442-445). Accordingly, in some embodiments of the present invention, the genome editing is achieved by a clustered regularly interspersed short palindromic repeats (CRISPR) system, a transcription activator-like effector nuclease (TALEN) system, or a zinc finger nuclease (ZFN) system.

One important type of genome editing is base editing. As used herein, the term “base editing” refers to a base mutation (substitution, deletion or addition) that causes point mutations in a target site within a target gene, with a few bases (one or two). Base editing can be distinguished from gene editing involving mutation of a relatively large number of bases. The base correction may be one that does not involve double-stranded DNA cleavage.

In some embodiments, the method further comprises selecting one or more of the identified alternative candidates for use in genome editing.

In some embodiments, the genome editing is achieved by a base editing technology using a DNA base editor or an RNA base editor.

In some embodiments, provided herein is a base editing technology according to the method of any of the preceding embodiments.

### Computer Readable Media (Software)

Any of the aforementioned methods of present disclosure may be implemented as computer program processes that are specified as a set of instructions recorded on a non-transitory computer-readable storage medium (also referred to as a computer-readable medium-CRM).

Accordingly, in still another aspect, provided herein is a non-transitory computer-readable storage medium storing one or more programs, the one or more programs comprising instructions, which when executed by one or more processors of an electronic device having a display, cause the electronic device to: a) receive a dataset of sequences from an input device, wherein the dataset of sequences comprises a reference sequence, a primary genetic variant of the reference sequence, and one or more secondary genetic variants in the reference sequence; b) automatically input the dataset of sequences to a trained machine-learning model to obtain one or more effect scores, wherein the model is configured to output one or more effect scores corresponding to the probabilities of one or more secondary genetic variants having a compensatory effect to the primary genetic variant, or the magnitudes of said effects; and c) display the predicted effect scores on a display device.

Examples of computer-readable storage media include RANI, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, ultra-density optical discs, any other optical or magnetic media, and floppy disks. In some embodiments, the computer-readable storage medium is a solid-state device, a hard disk, a CD-ROM, or any other non-volatile computer-readable storage medium.

The computer-readable storage media can store a set of computer-executable instructions (e.g., a “computer program”) that is executable by at least one processing unit and includes sets of instructions for performing various operations.

A computer program (also known as a program, software, software application, script, or code) can be written in any form of programming language, including compiled or interpreted languages, declarative or procedural languages, and it can be deployed in any form, including as a standalone program or as a module, component, or subroutine, object, or other component suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms or portions of code). A computer program can be deployed to be executed on one computer or on multiple computers that are located at one site or distributed across multiple sites and interconnected by a communication network. Examples of computer programs or computer code include machine code, such as is produced by a compiler, and files including higher-level code that are executed by a computer, an electronic component, or a microprocessor using an interpreter.

As used herein, the term “software” is meant to include firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Any combination of separate programs that together implement a software aspect described here is within the scope of the subject disclosure. In some implementations, the software programs, when installed to operate on one or more electronic systems, define one or more specific machine implementations that execute and perform the operations of the software programs.

Any suitable machine learning models may be used with the methods of the present invention and be implemented as computer program processes that are specified as a set of instructions recorded on a computer-readable storage medium. In some embodiments, the model is a discriminative model or a generative model.

### Electronic Devices

Further, any one of the preceding methods of the present disclosure may be implemented in one or more computer systems or other forms of apparatus. Examples of apparatus include but are not limited to, a computer, a tablet personal computer, a personal digital assistant, and a cellular telephone.

Accordingly, in still another aspect, provided herein is an electronic device, comprising: a display; one or more processors; a memory; and one or more programs, wherein the one or more programs are stored in the memory and configured to be executed by the one or more processors, the one or more programs including instructions for: a) receiving a dataset of sequences from an input device, wherein the dataset of sequences comprises a reference sequence, a primary genetic variant of the reference sequence, and one or more secondary genetic variants in the reference sequence; b) automatically inputting the dataset of sequences to a trained machine-learning model to obtain one or more effect scores, wherein the model is configured to output one or more effect scores corresponding to the probabilities of one or more secondary genetic variants having a compensatory effect to the primary genetic variant, or the magnitudes of said effects; and c) displaying the predicted effect scores on a display device.

In some embodiments, the electronic device may be a server computer, a client computer, a personal computer (PC), a user device, a tablet PC, a laptop computer, a personal digital assistant (PDA), a cellular telephone, or any machine capable of executing a set of instructions, sequential or otherwise, that specify actions to be taken by that machine. In some embodiments, the electronic device may further include keyboard and pointing devices, touch devices, display devices, and network devices.

As used herein, the terms “computer”, “processor”, and “memory” all refer to electronic or other technological devices. These terms exclude people or groups of people. For the purposes of the specification, the terms “display” or “displaying” means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device described herein for displaying information to the user and a virtual or physical keyboard and a pointing device, such as a finger, pencil, mouse or a trackball, by which the user can provide input to the computer. Other kinds of devices can be used to provide for interaction with a user as well; for example, feedback provided to the user can be any form of sensory feedback, such as visual feedback, auditory feedback, or tactile feedback; and input from the user can be received in any form, including acoustic, speed, or tactile input.

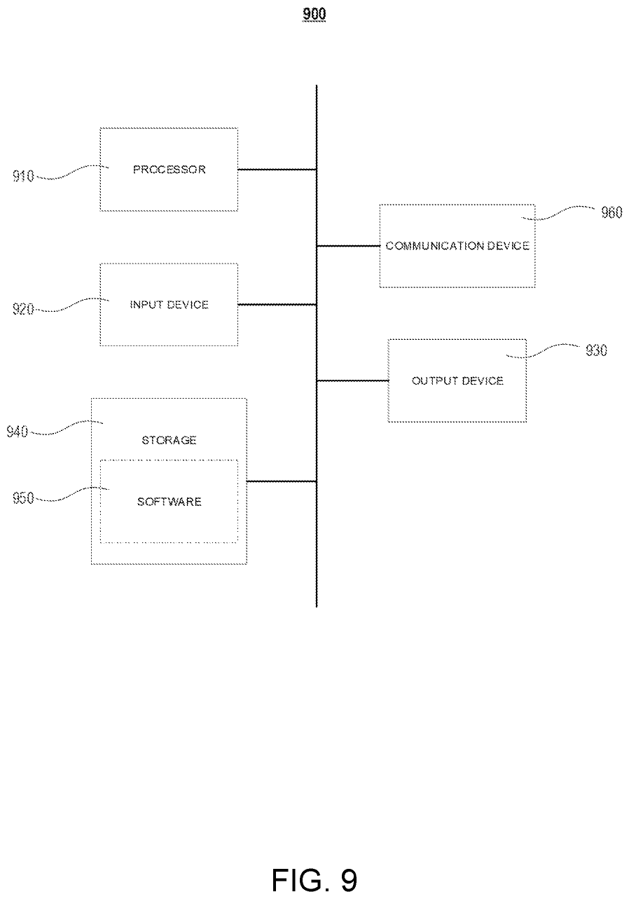

FIG. 9 illustrates an example of a computing device 900 in accordance with one embodiment. Device 900 can be a host computer connected to a network. Device 900 can be a client computer or a server. As shown in FIG. 9, device 900 can be any suitable type of microprocessor-based device, such as a personal computer, workstation, server or handheld computing device (portable electronic device) such as a phone or tablet. The device can include, for example, one or more of processor 910, input device 920, output device 930, storage 940, and a communication device 960. Input device 920 and output device 930 can generally correspond to those described above, and can be connectable or integrated with the computer.

Input device 920 can be any suitable device that provides input, such as a touch screen, keyboard or keypad, mouse, or voice-recognition device. Output device 930 can be any suitable device that provides output, such as a touch screen, haptics device, or speaker.

Storage 940 can be any suitable device that provides storage, such as an electrical, magnetic or optical memory including a RANI, cache, hard drive, or removable storage disk. Communication device 960 can include any suitable device capable of transmitting and receiving signals over a network, such as a network interface chip or device. The components of the computer can be connected in any suitable manner, such as via a physical bus or wirelessly.

Software 950, which can be stored in storage 940 and executed by processor 910, can include, for example, the programming that embodies the functionality of the present disclosure (e.g., as embodied in the devices as described above).

Software 950 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 940, that can contain or store programming for use by or in connection with an instruction execution system, apparatus, or device.

Software 950 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 900 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 900 can implement any operating system suitable for operating on the network. Software 950 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

### EXAMPLES

The following examples are offered to illustrate provided embodiments and are not intended to limit the scope of the present disclosure.

### Example 1: Using Machine Learning to Identify Compensatory Genetic Variants

This example illustrates a project aiming to use the methods described herein to identify compensatory genetic variants that may be useful to human genetics research and improvement of medicine.

The project focuses on two genes, BBS4 and RPGRIP1L, involved in ciliopathies, which are human disorders that arise from the dysfunction of motile and/or non-motile cilia. A deleterious and pathogenic primary genetic variant has been known in each of the two proteins—the N165H amino acid substitution in BBS4 and the R937L amino acid substitution in RPGRIP1L, which contributes to the Bardet-Biedl syndrome and Meckel-Gruber syndrome, respectively.

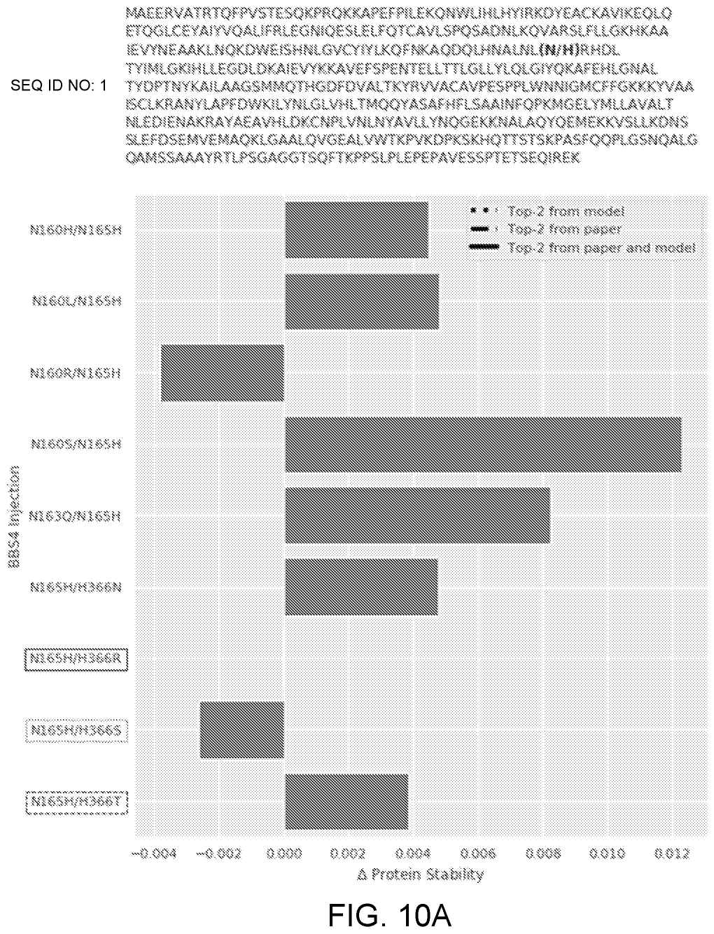

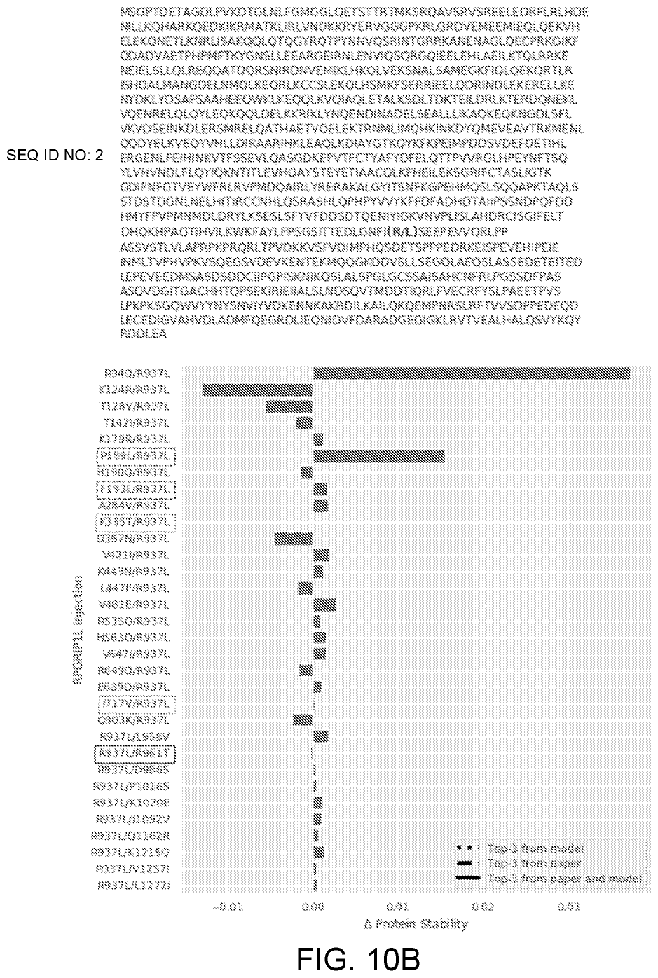

Using methods of the present disclosure, the project identified a number of candidate secondary genetic variants having a compensatory effort on the primary genetic variant in each of the two genes. FIG. 10A and FIG. 10B show results of the identification of compensatory genetic variants in the BBS4 protein (FIG. 10A) and RPGRIP1L protein (FIG. 10B).

For the BBS4 protein, the upper panel of FIG. 10A shows the polypeptide sequence of the BBS4 protein (SEQ ID NO: 1) with the primary genetic variant N/H variant in bold font at amino acid location 165. The lower panel of FIG. 10A shows a series of compensatory variant pairs including the N165H/H366R pair that produces one of the least differences in protein stability compared to the wild-type protein (i.e., lowest value in “Δ Protein Stability”), suggesting that the H366R variant has the highest likelihood to compensate for the deleterious primary genetic variant N165H in BBS4 protein that underpins the Bardet-Biedl syndrome.

For the RPGRIP1L protein, the upper panel of FIG. 10B shows the polypeptide sequence of the RPGRIP1L protein (SEQ ID NO: 2) with the primary genetic variant R/L, variant in bold font at amino acid location 937. The lower panel of FIG. 10B shows a series of compensatory mutation pairs including the R937L/R961 pair that produces one of the least differences in protein stability compared to the wild-type protein (i.e., lowest value in “Δ Protein Stability”), suggesting that the R961 variant has the highest likelihood to compensate for the deleterious primary genetic variant R937L in RPGRIP1L protein that underpins the Meckel-Gruber syndrome.

The identified candidate compensatory genetic variants were compared with those identified by Jordan et al. (Nature 524, 225-229 (2015), where a set of potential compensating mutations are investigated and functionally validated using the model organism zebrafish. Results show that, compared to the top-k candidates identified by Jordan et al. (with k=2 for BBS4 and k=3 for RPGRIP1L), at least one of the top-k compensating mutations identified by the methods of the present disclosure (shown in solid box in FIG. 10A and FIG. 10B) overlap with the findings from the zebrafish study.

Taken together, this example demonstrates successful implementation of using the methods of the present disclosure to identify compensatory genetic variants, which are useful for a variety of applications, such as being used as targets of genome editing and personalized medicine.

What is claimed is:

1. A method for assessing effects of genetic variants, comprising:
a) receiving a dataset of sequences from an input device,
wherein the dataset of sequences comprises a reference sequence, a primary genetic variant of the reference sequence, and one or more secondary genetic variants in the reference sequence;
b) automatically inputting the dataset of sequences to a trained machine-learning model to predict one or more effect scores,
wherein the model is configured to output one or more effect scores corresponding to the probabilities of one or more secondary genetic variants having a compensatory effect to the primary genetic variant, or the magnitudes of said effects; and
c) displaying the predicted effect scores on a display device.

2. The method of claim 1, wherein the model is trained by:
a) a pre-training task, comprising:
1) receiving a pre-training dataset comprising a plurality of batches of naturally occurring sequences;
2) inputting each batch of sequences into a language model, wherein the model is configured to output a pre-training set of semantic features; and
3) automatically updating the language model after each batch;
b) optionally, a fine-tuning task, comprising:
1) receiving a fine-tuning dataset comprising a plurality of batches of naturally occurring sequences,
wherein the fine-tuning dataset is a subset of the pre-training dataset, or a set of sequences that are related to the pre-training dataset by common ancestry, homology, or multiple sequence alignment;
2) inputting each batch of sequences into the language model, wherein the model is configured to output a fine-tuning set of semantic features; and
3) automatically updating the language model after each batch; and
c) a transfer learning task, comprising:
1) receiving a final training dataset comprising labeled sequences mapped to effects; and
2) training a neural network model based on the final training dataset, wherein the neural network model is configured to receive data corresponding to the pre-training set of semantic features and/or the fine-tuning set of semantic features, and output one or more effect scores.

3. The method of claim 1, wherein the model is trained by:
a) receiving a training dataset of sequences, comprising a training reference sequence and a training primary genetic variant, wherein the training primary genetic variant has an effect on the reference sequence with respect to a metric of interest;
b) inputting the training dataset into a generative procedure configured to generate one or more training secondary genetic variants according to a random seed;
c) calculating a loss function, wherein the loss function maps the combined effect of the primary and secondary genetic variants and the effect of the reference sequence onto a quantitative error score;
d) accepting or rejecting the one or more training secondary genetic variants according to one or more predetermined acceptance criteria on the loss function;
e) updating the generative procedure by incorporating the accepted one or more training secondary genetic variants in a new round of additional training secondary genetic variants; and
f) repeating steps b) to e) until the loss converges to a minimum.

4. The method of claim 3, wherein the true compensatory effect is obtained from a saturation mutagenesis analysis.

5. The method of claim 3, wherein the loss function is a binary loss function.

6. The method of claim 3, wherein the loss function is based on a distance metric.

7. The method of any one of claims 1-6, further comprising selecting one or more secondary genetic variants based on the effect scores.

8. The method of any one of claims 1-7, further comprising prioritizing one or more secondary genetic variants based on the effect scores.

9. The method of any one of claims 1-8, further comprising evaluating epistasis of one or more secondary genetic variants based on the effect scores.

10. The method of any one of claims 1-9, further comprising:
a) altering one or more of the secondary genetic variants in the genome of an organism;
b) identifying an impact of the alteration on an endophenotype, wherein the endophenotype is a quantifiable phenotype at a sub-organismal level that can be measured by a biochemical, gene expression, or protein level assay, or visually via microscopy; and
c) updating the model using the identified endophenotypic impact.

11. The method of any one of claims 1-10, wherein the genetic variant is an allele or a mutation as compared to the reference sequence.

12. The method of any one of claims 1-11, wherein the primary genetic variant is a deleterious genetic variant having a deleterious or disease-causing effect as compared to the reference sequence.

13. The method of any one of claims 1-12, wherein the primary genetic variant is a beneficial genetic variant having a beneficial or disease-preventing effect as compared to the reference sequence.

14. The method of any one of claims 1-13, wherein the dataset of sequences are clustered by sequence similarity.

15. The method of any one of claims 1-14, wherein the dataset of sequences are obtained from a sequence database.

16. The method of claim 15, wherein the sequence database is the UniRef database, the UniParc database, the UniProt database, the Pfam database, or the SwissProt database.

17. The method of any one of claims 1-16, wherein the dataset of sequences are DNA sequences, RNA sequences, or protein sequences.

18. The method of any one of claims 1-17, wherein the dataset of sequences are sequences from a single gene or a protein encoded thereby.

19. The method of any one of claims 1-18, wherein the dataset of sequences are sequences from a single gene family or a protein family encoded thereby.

20. The method of any one of claims 1-19, wherein the dataset of sequences are sequences from different genes or proteins encoded thereby, wherein the encoded proteins physically interact to form a complex.

21. The method of any one of claims 1-20, wherein the dataset of sequences are sequences from different components within a virus, an organelle, a cell, a tissue, an organ, or an organism.

22. The method of any one of claims 1-21, wherein the dataset of sequences are viral sequences, bacterial sequences, algal sequences, fungal sequences, plant sequences, animal sequences, human sequences, or sequences from a particular phylogenetic lineage.

23. The method of any one of claims 1-22, wherein the dataset of sequences are from one or more coronaviruses.

24. The method of any one of claims 1-23, wherein the dataset of sequences are from one or more cancer cells.

25. The method of any one of claims 1-24, wherein the effect is an effect at a molecular level, a cellular level, a sub-organismal level, or an organismal level.

26. The method of any one of claims 1-25, wherein the effect is an effect affecting an endophenotype selected from a group consisting of messenger RNA (mRNA) abundance, gene transcript splicing ratio, protein abundance, micro RNA (miRNA) or small RNA (siRNA) abundance, translational efficiency, ribosome occupancy, protein modification, metabolite abundance, allele specific expression (ASE), or visual trait measured at the sub-organismal level.

27. The method of any one of claims 1-26, wherein the effect is an effect affecting a protein property.

28. The method of any one of claims 1-27, wherein the effect is an effect affecting protein structure, protein conformation, protein molecular or cellular function, protein stability, protein solvent accessibility, enzymatic affinity, or enzymatic efficiency.

29. The method of any one of claims 1-28, wherein the effect is a collection of effects characterizing the state of a protein.

30. The method of any one of claims 1-29, wherein the effect is an effect affecting fitness of an organism.

31. The method of any one of claims 1-30, wherein the effect is interpretable to humans and/or machines.

32. A method for designing a molecule with a desired effect, comprising:
a) receiving a dataset of sequences from an input device,
wherein the dataset of sequences comprises a reference sequence, a primary genetic variant of the reference sequence, and one or more secondary genetic variants in the reference sequence;
b) automatically inputting the dataset of sequences to a trained machine-learning model to predict one or more effect scores,
wherein the model is configured to output one or more effect scores corresponding to the probabilities of one or more secondary genetic variants having a compensatory effect to the primary genetic variant, or the magnitudes of said effects;
c) displaying the predicted effect scores on a display device; and
d) designing a molecule based on the effect scores.

33. The method of claim 32, further comprising synthesizing the designed molecule.

34. The method of any one of claims 32-33, wherein the effect of the designed molecule is stability, solubility, affinity, biological activity, bioavailability, a chemical property, a physical property, or a structural property.

35. The method of any one of claims 32-34, wherein the designed molecule is a DNA molecule, an RNA molecule, or a protein molecule.

36. The method of any one of claims 32-35, wherein the designed molecule is a single stranded DNA (ssDNA) or a double stranded DNA (dsDNA).

37. The method of any one of claims 32-36, wherein the designed molecule is a messenger RNA (mRNA), a transfer RNA (tRNA), a ribosomal RNA (rRNA), a small RNA (sRNA), or a guide RNA (gRNA).

38. The method of any one of claims 32-37, wherein the designed molecule is an antibody, a contractile protein, an enzyme, a hormonal protein, a structural protein, a storage protein, or a transport protein.

39. The method of any one of claims 32-38, wherein the designed molecule is a viral molecule, a bacterial molecule, an algal molecule, a fungal molecule, a plant molecule, an animal molecule, or a human molecule.

40. The method of claim 39, wherein the designed molecule is a virus protein.

41. The method of claim 40, wherein the virus protein is a protein from a coronavirus.

42. The method of claim 41, wherein the coronavirus is a severe acute respiratory syndrome coronavirus 2 (SARS-CoV-2) that is the causal agent for the infectious disease coronavirus disease 2019 (COVID-19).

43. A method for providing personalized and probabilistic information for a patient, comprising:
a) receiving a dataset of sequences associated with a patient from an input device, wherein the dataset of sequences comprises a reference sequence, a primary genetic variant of the reference sequence, and one or more secondary genetic variants in the reference sequence;
b) automatically inputting the dataset of sequences to a trained machine-learning model to predict one or more effect scores,
wherein the model is configured to output one or more effect scores corresponding to the probabilities of one or more secondary genetic variants having a compensatory effect to the primary genetic variant, or the magnitudes of said effects;
c) displaying the predicted effect scores on a display device; and
d) assisting in selection of one or more medical choices specific to the patient based on the effect scores.

44. The method of claim 43, wherein the attribute associated with the patient is selected from the group consisting of genetic profile, predisposition or response to a disease, and response to a treatment.

45. The method of claim 44, wherein the genetic profile is from one or more cancer tumors of the patient.

46. The method of claim 44, wherein the disease is selected from the group consisting of cancer, obesity, hypertension, a cardiovascular disease, an infectious disease, an autoimmune disease, a genetic disease, a liver disease, insulin resistance, Crohn's disease, dementia, Alzheimer's disease, cerebral infarction, hemophilia, viral hepatitis, sickle cell disease, multiple sclerosis, and muscular dystrophy.

47. The method of claim 44, wherein the treatment is selected from the group consisting of drug administration, chemotherapy, radiation therapy, immunotherapy, and gene therapy.

48. The method of any one of claims 43-47, wherein the one or more medical choices are selected from the group consisting of prognosis, diagnosis, treatment, intervention, and prevention.

49. A method for predicting resistance of a pathogen to an anti-pathogen treatment, comprising:
a) receiving a dataset of sequences associated with a pathogen from an input device, wherein the dataset of sequences comprises a reference sequence, a primary genetic variant of the reference sequence, and one or more secondary genetic variants in the reference sequence;
b) automatically inputting the dataset of sequences to a trained machine-learning model to predict one or more effect scores,
wherein the model is configured to output one or more effect scores corresponding to the probabilities of one or more secondary genetic variants having a compensatory effect to the primary genetic variant, or the magnitudes of said effects,
wherein the effect affects an attribute associated with the pathogen having resistance to an anti-pathogen treatment; and
c) displaying the predicted effect scores on a display device, corresponding to the predicted resistance of the pathogen to the anti-pathogen treatment.

50. The method of claim 49, wherein the pathogen is a virus, a prion, a viroid, a bacterium, a fungus, a protozoan, or a parasite.

51. The method of any one of claims 49-50, wherein the attribute associated with the pathogen is selected from the group consisting of nucleic acid replication, DNA integration into a host genome, gene expression, protein synthesis, metabolism, cell membrane synthesis, cell wall synthesis, and peptidoglycan biosynthesis.

52. The method of any one of claims 49-51, wherein the anti-pathogen treatment is administering a drug selected from the group consisting of an antiviral, an antibacterial, an antibiotic, an antifungal, an antiparasitic, and a pesticide.

53. The method of any one of claims 49-52, wherein the pathogen is Neisseria gonorrhea and the anti-pathogen treatment is administration of ciprofloxacin or ceftriaxone.

54. A method for identifying targets for genetically improving a trait in an organism, comprising:
a) receiving a dataset of sequences associated with an organism from an input device, wherein the dataset of sequences comprises a reference sequence, a primary genetic variant of the reference sequence, and one or more secondary genetic variants in the reference sequence;
b) automatically inputting the dataset of sequences to a trained machine-learning model to predict one or more effect scores,
wherein the model is configured to output one or more effect scores corresponding to the probabilities of one or more secondary genetic variants having a compensatory effect to the primary genetic variant, or the magnitudes of said effects, and
wherein the effect affects an attribute associated with a trait of the organism; and
c) displaying the predicted effect scores on a display device, corresponding to the targets for genetically improving the trait in the organism.

55. The method of claim 54, further comprising selecting one or more of the identified targets for genetic improvement of the organism.

56. The method of any one of claims 54-55, further comprising selecting an organism with the improved trait.

57. The method of any one of claims 54-56, wherein the genetic improvement is achieved by conventional breeding.

58. The method of any one of claims 54-57, wherein the genetic improvement is achieved by a transgenic technology or a genome editing technology.

59. The method of claim 58, wherein the genome editing technology is a base editing technology using a DNA base editor or an RNA base editor.

60. The method of any one of claims 54-59, wherein the genome editing is achieved by a clustered regularly interspersed short palindromic repeats (CRISPR) system, a transcription activator-like effector nuclease (TALEN) system, or a zinc finger nuclease (ZFN) system.

61. The method of claim 60, wherein the genome editing is achieved by coupling with a recombination system.

62. The method of claim 61, wherein the recombination system is a lambda phage derived recombination (lambda Red) system.

63. The method of any one of claims 54-62, wherein the organism is maize, wheat, barley, oat, rice, soybean, oil palm, safflower, sesame, tobacco, flax, cotton, sunflower, pearl millet, foxtail millet, sorghum, canola, cannabis, a vegetable crop, a forage crop, an industrial crop, a woody crop, or a biomass crop.

64. The method of claim 63, wherein the trait is yield, overall fitness, biomass, photosynthetic efficiency, nutrient use efficiency, heat tolerance, drought tolerance, herbicide tolerance, or disease resistance.

65. The method of any one of claims 54-63, wherein the organism is cattle, sheep, goat, horse, pig, chicken, duck, goose, rabbit, or fish.

66. The method of claim 65, wherein the trait of the organism is growth rate, feed use efficiency, meat yield, meat quality, milk yield, milk quality, egg yield, egg quality, wool yield, or wool quality.

67. An organism genetically improved by the method of any one of claims 54-66.

68. A method for identifying genetic variants as alternative candidates for use as targets that are more easily accessible by a transgenic technology or a genome editing technology, comprising:
a) receiving a dataset of sequences associated with an organism from an input device, wherein the dataset of sequences comprises a reference sequence, a primary genetic variant of the reference sequence, and two or more secondary genetic variants in the reference sequence;
b) automatically inputting the dataset of sequences to a trained machine-learning model to predict one or more effect scores,
wherein the model is configured to output one or more effect scores corresponding to the probabilities of one or more secondary genetic variants having a compensatory effect to the primary genetic variant, or the magnitudes of said effects; and
c) displaying the predicted effect scores on a display device, corresponding to the genetic variants as alternative candidates for use as more accessible targets in genome editing.

69. The method of claim 68, further comprising producing the genetic variants identified as alternative candidates targets in genome editing.

70. The method of any one of claims 68-69, wherein the genome editing is achieved by a base editing technology using a DNA base editor or an RNA base editor.

71. A base editing technology according to the method of claim 70.

72. A non-transitory computer-readable storage medium storing one or more programs, the one or more programs comprising instructions, which when executed by one or more processors of an electronic device having a display, cause the electronic device to:
a) receive a dataset of sequences from an input device,
wherein the dataset of sequences comprise a reference sequence, a primary genetic variant of the reference sequence, and one or more secondary genetic variants in the reference sequence;
b) automatically input the dataset of sequences to a trained machine-learning model to predict one or more effect scores,
wherein the model is configured to output one or more effect scores corresponding to the probabilities of one or more secondary genetic variants having a compensatory effect to the primary genetic variant, or the magnitudes of said effects; and
c) display the predicted effect scores on a display device.

73. The computer-readable medium of claim 72, wherein the model is a discriminative model or a generative model.

74. An electronic device, comprising:
a display;
one or more processors;
a memory; and
one or more programs, wherein the one or more programs are stored in the memory and configured to be executed by the one or more processors, the one or more programs including instructions for:
a) receiving a dataset of sequences from an input device,
wherein the dataset of sequences comprise a reference sequence, a primary genetic variant of the reference sequence, and one or more secondary genetic variants in the reference sequence;
b) automatically inputting the dataset of sequences to a trained machine-learning model to predict one or more effect scores,
wherein the model is configured to output one or more effect scores corresponding to the probabilities of one or more secondary genetic variants having a compensatory effect to the primary genetic variant, or the magnitudes of said effects; and
c) displaying the predicted effect scores on a display device.


SEQUENCE LISTING (2 sequences)

SEQ ID NO: 1 - protein, 519 aa, Homo sapiens
  VARIANT 165: Xaa = Asn or His
MAEERVATRTQFPVSTESQKPRQKKAPEFPILEKQNWLIHLHYIRKDYEACKAVIKEQLQETQGLCEYAIYVQALIFRLEGNIQESLELFQTCAVLSPQSADNLKQVARSLFLLGKHKAAIEVYNEAAKLNQKDWEISHNLGVCYIYLKQFNKAQDQLHNALNLXRHDLTYIMLGKIHLLEGDLDKAIEVYKKAVEFSPENTELLTTLGLLYLQLGIYQKAFEHLGNALTYDPTNYKAILAAGSMMQTHGDFDVALTKYRVVACAVPESPPLWNNIGMCFFGKKKYVAAISCLKRANYLAPFDWKILYNLGLVHLTMQQYASAFHFLSAAINFQPKMGELYMLLAVALTNLEDIENAKRAYAEAVHLDKCNPLVNLNYAVLLYNQGEKKNALAQYQEMEKKVSLLKDNSSLEFDSEMVEMAQKLGAALQVGEALVWTKPVKDPKSKHQTTSTSKPASFQQPLGSNQALGQAMSSAAAYRTLPSGAGGTSQFTKPPSLPLEPEPAVESSPTETSEQIREK

SEQ ID NO: 2 - protein, 1315 aa, Homo sapiens
  VARIANT 937: Xaa = Arg or Leu
MSGPTDETAGDLPVKDTGLNLFGMGGLQETSTTRTMKSRQAVSRVSREELEDRFLRLHDENILLKQHARKQEDKIKRMATKLIRLVNDKKRYERVGGGPKRLGRDVEMEEMIEQLQEKVHELEKQNETLKNRLISAKQQLQTQGYRQTPYNNVQSRINTGRRKANENAGLQECPRKGIKFQDADVAETPHPMFTKYGNSLLEEARGEIRNLENVIQSQRGQIEELEHLAEILKTQLRRKENEIELSLLQLREQQATDQRSNIRDNVEMIKLHKQLVEKSNALSAMEGKFIQLQEKQRTLRISHDALMANGDELNMQLKEQRLKCCSLEKQLHSMKFSERRIEELQDRINDLEKERELLKENYDKLYDSAFSAAHEEQWKLKEQQLKVQIAQLETALKSDLTDKTEILDRLKTERDQNEKLVQENRELQLQYLEQKQQLDELKKRIKLYNQENDINADELSEALLLIKAQKEQKNGDLSFLVKVDSEINKDLERSMRELQATHAETVQELEKTRNMLIMQHKINKDYQMEVEAVTRKMENLQQDYELKVEQYVHLLDIRAARIHKLEAQLKDIAYGTKQYKFKPEIMPDDSVDEFDETIHLERGENLFEIHINKVTFSSEVLQASGDKEPVTFCTYAFYDFELQTTPVVRGLHPEYNFTSQYLVHVNDLFLQYIQKNTITLEVHQAYSTEYETIAACQLKFHEILEKSGRIFCTASLIGTKGDIPNFGTVEYWFRLRVPMDQAIRLYRERAKALGYITSNFKGPEHMQSLSQQAPKTAQLSSTDSTDGNLNELHITIRCCNHLQSRASHLQPHPYVVYKFFDFADHDTAIIPSSNDPQFDDHMYFPVPMNMDLDRYLKSESLSFYVFDDSDTQENIYIGKVNVPLISLAHDRCISGIFELTDHQKHPAGTIHVILKWKFAYLPPSGSITTEDLGNFIXSEEPEVVQRLPPASSVSTLVLAPRPKPRQRLTPVDKKVSFVDIMPHQSDETSPPPEDRKEISPEVEHIPEIEINMLTVPHVPKVSQEGSVDEVKENTEKMQQGKDDVSLLSEGQLAEQSLASSEDETEITEDLEPEVEEDMSASDSDDCIIPGPISKNIKQSLALSPGLGCSSAISAHCNFRLPGSSDFPASASQVDGITGACHHTQPSEKIRIEIIALSLNDSQVTMDDTIQRLFVECRFYSLPAEETPVSLPKPKSGQWVYYNYSNVIYVDKENNKAKRDILKAILQKQEMPNRSLRFTVVSDPPEDEQDLECEDIGVAHVDLADMFQEGRDLIEQNIDVFDARADGEGIGKLRVTVEALHALQSVYKQYRDDLEA